(19) United States
(12) Patent Application Publication
(10) Pub. No.: US 2023/0091297 A1
(43) Pub. Date: Mar. 23, 2023

(54) INHIBITORS OF HSP70 PROTEINS

(71) Applicant: ARIZONA BOARD OF REGENTS ON BEHALF OF THE UNIVERSITY OF ARIZONA, Tucson, AZ (US)

(72) Inventors: Eli Chapman, Tucson, AZ (US); Andrew J. Ambrose, Tucson, AZ (US); Taoda Shi, Tucson, AZ (US)

(21) Appl. No.: 17/791,167

(22) PCT Filed: Mar. 12, 2021
(86) PCT No.: PCT/US21/22194

§ 371 Date: Jul. 6, 2022

Related U.S. Application Data
(60) Provisional application No. 62/989,247, filed on Mar. 13, 2020.

(51) Int. Cl. A61K 31/426; A61K 31/427; A61K 31/428; A61P 35/00

(52) U.S. Cl. CPC A61K 31/426; A61K 31/427; A61K 31/428; A61P 35/00

(57) ABSTRACT

Provided are compounds useful for selectively inhibiting HSP70 isoforms. Also provided are methods of inhibiting HSP70 proteins and methods of treating a disease characterized by overexpression of a HSP70, such as cancer. In particular embodiments, the disclosed compounds may be used as potent inhibitors for HSPA5 and may display greater than 20-fold selectivity over other HSP70 isoforms.

### CROSS-REFFERENCE To RELATED APPLICATION

This application claims the benefit of and priority to U.S. Provisional Application No. 62/989,247, filed on Mar. 13, 2020, the entire content of all of which is hereby incorporated by reference.

### BACKGROUND

The heat shock protein 70 (HSP70) family of proteins are some of the most highly conserved proteins in all of biology, and all organisms contain at least one HSP70 protein. HSP70 proteins have myriad functions but are generally thought of as holdases that help prevent aggregation of newly translated or misfolded proteins. In particular, HSP70 proteins may be targets for anti-cancer and neurodegenerative therapies.

To date, there are no HSP70 inhibitors that have been approved for treatment of diseases in humans. The human HSP70 isoforms are highly conserved from both a structural and functional perspective, but a few selective, if not completely specific molecules do exist. There are many possible reasons for a lack of clinical success of these molecules including numerous pharmacokinetic and metabolic liabilities. Another possible explanation could be a lack of selectivity for specific isoforms, as most inhibitors of an HSP70 also inhibit the constitutive HSP70 (HSC70 or HSPA8). In general, it is thought to be important to avoid inhibiting HSC70, as this protein is essential in mice due to its numerous housekeeping functions. However, mice with their heat inducible HSP70 isoforms knocked out are healthy, but susceptible to stress conditions. Therefore, any HSP70 inhibitor should avoid activity against HSPA8 which happens to be perhaps the most difficult HSP70 to avoid. Thus, there remains a need for alternative HSP70 inhibitors, especially those with high selectivity for particular HSP70 isoforms.

### SUMMARY

In one aspect, the present disclosure provides a compound of formula (I′), or a pharmaceutically acceptable salt thereof,

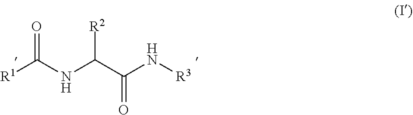

wherein

R¹′ is an aryl or a heteroaryl, wherein R¹′ is optionally substituted with one or more R^{a}′;

R²′ is C₁₋₆ alkyl, aryl, heteroaryl, C₁₋₄ alkylene-aryl, or C₁₋₄ alkylene-heteroaryl, wherein R²′ is optionally substituted with one or more R^{b}′;

R³′ is an aryl or a heteroaryl, wherein R³′ is optionally substituted with one or more R^{c}′;

R^{a}′, R^{b}′, and R^{c}′ at each occurrence are independently halogen, —CN, nitro, N₃, —SO₂NH₂, or —Y—R^{Y};

Y is bond, O, NH, C(O), OC(O), C(O)NH, or S; and

R^{Y} is H, alkyl, alkenyl, alkynyl, alkoxy, cycloalkyl, aryl, heteroaryl, or heterocyclyl, wherein R^{Y} is optionally substituted;

provided that the compound is not N-(3-methyl-1-oxo-1-(thiazol-2-ylamino)butan-2-yl)thiophene-2-carboxamide.

The present disclosure also provides a pharmaceutical composition comprising a compound as disclosed herein, or a pharmaceutically acceptable salt thereof, and a pharmaceutically acceptable carrier.

In another aspect, the present disclosure provides a method for inhibiting a heat shock protein 70 (HSP70) comprising contacting the HSP70 with a pharmaceutically effective amount of a compound of formula (I), or a pharmaceutically acceptable salt thereof,

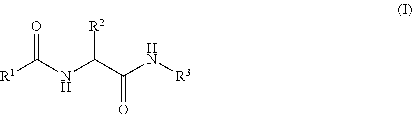

wherein

R¹ is an aryl or a heteroaryl, wherein R¹ is optionally substituted with one or more R^{a};

R² is C₁₋₆ alkyl, aryl, heteroaryl, C₁₋₄ alkylene-aryl, or C₁₋₄ alkylene-heteroaryl, wherein R² is optionally substituted with one or more R^{b};

R³ is an aryl or a heteroaryl, wherein R³ is optionally substituted with one or more R^{c};

R^{a}, R^{b}, and R^{c} at each occurrence are independently halogen, —CN, nitro, N₃, —SO₂NH₂, or —X—R^{X},

X is bond, O, NH, C(O), OC(O), C(O)NH, or S; and

R^{X} is H, alkyl, alkenyl, alkynyl, alkoxy, cycloalkyl, aryl, heteroaryl, or heterocyclyl, wherein R^{X} is optionally substituted.

In yet another aspect, the present disclosure provides a method for treating a disease characterized by overexpression of a heat shock protein 70 (HSP70) comprising administrating to a subject in need thereof a pharmaceutically effective amount of a compound of formula (I), or a pharmaceutically acceptable salt thereof,

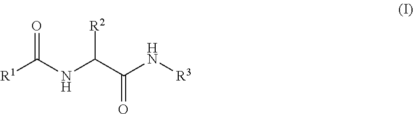

wherein

R¹ is an aryl or a heteroaryl, wherein R¹ is optionally substituted with one or more R^{a};

R² is C₁₋₆alkyl, aryl, heteroaryl, C₁₋₄alkylene—aryl, or C₁₋₄alkylene-heteroaryl, wherein R² is optionally substituted with one or more R^{b};

R³ is an aryl or a heteroaryl, wherein R³ is optionally substituted with one or more R^{c};

R^{a}, R^{b}, and R^{c} at each occurrence are independently halogen, —CN, nitro, N₃, —SO₂NH₂, or —X—R^{X},

X is bond, O, NH, C(O), OC(O), C(O)NH, or S; and

R^{X} is H, alkyl, alkenyl, alkynyl, alkoxy, cycloalkyl, aryl, heteroaryl, or heterocyclyl, wherein R^{X} is optionally substituted.

### BRIEF DESCRIPTION OF THE DRAWINGS

FIGS. 1A and 1B show the result of a screen of 70,000 compounds, which reveals compound 1 as a potent disruptor of the HSPA5-peptide interaction. FIG. 1A shows Z′ factor from FP based HSPA5-NRLLLTG interaction in 384 well plates. FIG. 1B shows dose response of compound 1.

FIG. 2A shows the effect of chirality in activity of compound 1. The S enantiomer of 1 is twice as potent as 1 and the R enantiomer has no detectible activity. *p<0.05, #IC50>100. FIG. 2B shows the effect of amide bond in the activity of 1. Reduction or methylation of the amide bond in 1 completlely ablates inhibitory activity of 1.

FIGS. 3A and 3B show that compound 2 binds to the SBD of HSPA5, but not in an identical manner as the NR peptide. FIG. 3A shows inhibition of NR binding to HSPA5 by 2. FIG. 3B shows stimulation of ATPase activity of HSPA5 by either 2 or NRLLLTG peptide. *p<0.05.

FIGS. 4A-4C show the activity of compound 2 with a panel of human HSP70 SBDs. FIG. 4A shows the binding affinity of NRLLLTG and ALLLSAPRR to HSP70 substrate biding domains. FIG. 4B shows the Z′ factor for NRLLLTG and ALLLSAPRR with HSP70 SBDs in 384 well plates. FIG. 4C shows the dose response of 2 with HSP70 SBDs. #: IC₀>50 μM.

FIG. 5 shows the IC₅₀ of compound 8 for HSPA5 (dose response of 8 against 250 nM HSPA5).

FIG. 6 shows the results of kinetic binding of NR and P1 peptides to HSP70 SBD. FP was measured every five minutes for 2 hours after the addition of fluorescent peptide to a fixed concentration of HSP70. Error bars represent the standard deviation of 3 independent measurements.

FIG. 7 shows the results of peptide independent binding of compound 22 to 7 HSP70 SBDs. FP based binding of 22 to each canonical HSP70 SBD. # Kd>25 μM.

FIG. 8 shows NCI-60 cancer panel results, which include the activity of compound 8 against 60 cancer cell lines at 10 μM.

FIGS. 9A and 9B show the activity of compound 8 in cancer cell lines. FIG. 9A shows ZIP synergy between the p97 inhibitor CB5083 and 8. FIG. 9B shows the correlation between 8 and the known HSPA5 inhibitor HA15 in a growth inhibition assay in various cancer cell lines.

FIG. 10 shows representative results of compound 24 and NRLLLTG peptide in blocking the ability of HspA5 to break up IRE1 dimers.

FIG. 11 shows an overlay of ⁵N-HSQC spectra of HspA5 substrate biding domain (SBD) and HspA5 SBD bound to compound 24.

FIG. 12 shows the synergy between compound 8 and various ER stress inducers. ** indicates a p-value of <0.01 between a given treatment with and without 10 μM of 8.

### DETAILED DESCRIPTION

A recent study has illustrated that a peptide binding fluorescence polarization assay can be used to identify selective, substrate-competitive inhibitors of HSP70 isoforms. The present disclosure describes the application of that assay to 70,000 compounds and the expansion of the assay to other human HSP70 isoforms. This led to the discovery of a drug-like amino acid-based inhibitor with reasonable specificity for the endoplasmic reticular HSP70, HSPA5 (GRP78). Under a medicinal chemistry approach, various compounds were prepared through SAR studies in parallel assays for six HSP70 isoforms, and the results herein illustrate the potency and selectivity of the present compounds. As a non-limiting example, compound 8 of the present disclosure has a strong positive correlation in cytotoxicity analysis with HA15, a known and selective HSPA5 inhibitor. In a particular embodiment, compound 8 has strong positive synergy with CB5083, a p97 inhibitor known to induce ER stress without increasing HSPA5 levels.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

The term “alkyl” as used herein, means a straight or branched, saturated hydrocarbon chain containing from 1 to 20 carbon atoms. The term “lower alkyl” or “C₁₋₆alkyl” means a straight or branched chain hydrocarbon containing from 1 to 6 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl” as used herein, means an unsaturated hydrocarbon chain containing from 2 to 20 carbon atoms and at least one carbon-carbon double bond.

The term “alkynyl” as used herein, means an unsaturated hydrocarbon chain containing from 2 to 20 carbon atoms and at least one carbon-carbon triple bond.

The term “alkylene”, as used herein, refers to a divalent group derived from a straight or branched chain hydrocarbon of 1 to 10 carbon atoms, for example, of 2 to 5 carbon atoms. Representative examples of alkylene include, but are not limited to, —CH₂CH₂—, —CH₂CH₂CH₂—, —CH₂CH₂CH₂CH₂—, and —CH₂CH₂CH₂CH₂CH₂—.

The term “aryl” as used herein, refers to a phenyl group, or a bicyclic fused ring system. Bicyclic fused ring systems are exemplified by a phenyl group appended to the parent molecular moiety and fused to a cycloalkyl group, as defined herein, a phenyl group, a heteroaryl group, as defined herein, or a heterocycle, as defined herein. Representative examples of aryl include, but are not limited to, indolyl, naphthyl, phenyl, quinolinyl and tetrahydroquinolinyl.

The term “haloalkyl” as used herein, means an alkyl group, as defined herein, in which one, two, three, four, five, six, seven or eight hydrogen atoms are replaced by a halogen. Representative examples of haloalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “cycloalkyl” as used herein, means a monovalent group derived from an all-carbon ring system containing zero heteroatoms as ring atoms, and zero double bonds. The all-carbon ring system can be a monocyclic, bicylic, or tricyclic ring system, and can be a fused ring system, a bridged ring system, or a spiro ring system, or combinations thereof. Examples of cycloalkyls include, but are not limited to, cyclopropyl, cyclobutyl, cyclopentyl, cyclohexyl, cycloheptyl, cyclooctyl, and

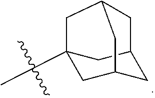 [SMILES: *C12CC3CC(CC(C3)C1)C2]

The cycloalkyl groups described herein can be appended to the parent molecular moiety through any substitutable carbon atom.

The term “cycloalkenyl” as used herein, means a non-aromatic monocyclic or multicyclic ring system containing at least one carbon-carbon double bond and preferably having from 5-10 carbon atoms per ring. Exemplary monocyclic cycloalkenyl rings include cyclopentenyl, cyclohexenyl or cycloheptenyl.

The term “halogen” as used herein, means Cl, Br, I, or F.

The term “heteroaryl” as used herein, refers to an aromatic monocyclic ring or an aromatic bicyclic ring system or an aromatic tricyclic ring system. The aromatic monocyclic rings are five or six membered rings containing at least one heteroatom independently selected from the group consisting of N, O, and S (e.g. 1, 2, 3, or 4 heteroatoms independently selected from O, S, and N). The five membered aromatic monocyclic rings have two double bonds and the six membered six membered aromatic monocyclic rings have three double bonds. The bicyclic heteroaryl groups are exemplified by a monocyclic heteroaryl ring appended to the parent molecular moiety and fused to a monocyclic cycloalkyl group, as defined herein, a monocyclic aryl group, as defined herein, a monocyclic heteroaryl group, as defined herein, or a monocyclic heterocycle, as defined herein. The tricyclic heteroaryl groups are exemplified by a monocyclic heteroaryl ring appended to the parent molecular moiety and fused to two of a monocyclic cycloalkyl group, as defined herein, a monocyclic aryl group, as defined herein, a monocyclic heteroaryl group, as defined herein, or a monocyclic heterocycle, as defined herein. Representative examples of monocyclic heteroaryl include, but are not limited to, pyridinyl (including pyridin-2-yl, pyridin-3-yl, pyridin-4-yl), pyrimidinyl, pyrazinyl, thienyl, furyl, thiazolyl, thiadiazolyl, isoxazolyl, pyrazolyl, and 2-oxo-1,2-dihydropyridinyl. Representative examples of bicyclic heteroaryl include, but are not limited to, chromenyl, benzothienyl, benzodioxolyl, benzotriazolyl, quinolinyl, thienopyrrolyl, thienothienyl, imidazothiazolyl, benzothiazolyl, benzofuranyl, indolyl, quinolinyl, imidazopyridine, benzooxadiazolyl, and benzopyrazolyl. Representative examples of tricyclic heteroaryl include, but are not limited to, dibenzofuranyl and dibenzothienyl. The monocyclic, bicyclic, and tricyclic heteroaryls are connected to the parent molecular moiety through any carbon atom or any nitrogen atom contained within the rings.

The term “heterocycle” or “heterocyclic” as used herein, means a monocyclic heterocycle, a bicyclic heterocycle, or a tricyclic heterocycle. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, 1,3-dimethylpyrimidine-2,4(1H,3H)-dione, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, 2-azaspiro[3.3]heptan-2-yl, azabicyclo[2.2.1]heptyl (including 2-azabicyclo[2.2.1]hept-2-yl), 2,3-dihydro-1H-indolyl, isoindolinyl, octahydrocyclopenta[c]pyrrolyl, octahydropyrrolopyridinyl, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.1^{3,7}]decane), and oxa-adamantane (2-oxatricyclo[3.3.1.1^{3,7}]decane). The monocyclic, bicyclic, and tricyclic heterocycles are connected to the parent molecular moiety through any carbon atom or any nitrogen atom contained within the rings.

The hydroxyl, amino, or carboxyl group as disclosed herein may be protected by a protecting group. The term “protecting group” refers to a moiety that prevents chemical reactions from occurring on a heteroatom (such as, N, O, or S) to which that protecting group is attached. The protected groups may be de-protected to provide, for example, a —OH, —NH₂, or —C(O)OH group. The term “protected amino,” “protected hydroxyl,” or “protected carboxyl” means a group resulting from the attachment of a suitable protecting group to an amino, a hydroxyl, or acarboxyl group, respectively. The term “amino proctecing group,” “hydroxyl protecting group,” or “carboxyl protecting group” refers to a group suitable for protecting an amino, a hydroxyl, or a carboxyl, respectively. Various protecting groups are well known in the art and include those described in detail in Greene's Protective Groups in Organic Synthesis, T. W. Greene and P. G. M. Wuts, 5th edition, John Wiley & Sons, 2014, the entirety of which is incorporated herein by reference. For example, suitable amino protecting groups include, but are not limited to, carbobenzyloxy (Cbz); t-butyloxycarbonyl (Boc); 9-fluorenylmethyloxycarbonyl (Fmoc), 2,2,2-trichloroethyloxycarbonyl (Troc), and allyloxycarbonyl (Alloc). In each of the foregoing, the —NH— represents the nitrogen from the amino group that is being protected. Suitable hydroxyl protecting groups include, but are not limited to, methoxymethyl ether (MOM), tetrahydropyranyl ether (THP), t-butyl ether, allyl ether, benzyl ether, trimethylsilyl (TMS), t-butyldimethylsilyl (TBDMS), acetyl, benzoyl, and pivalic acid ester. Suitable carboxyl protecting groups include, but are not limited to, methyl ester, t-butyl ester, and benzyl ester

In some instances, the number of carbon atoms in a hydrocarbyl substituent (e.g., alkyl or cycloalkyl) is indicated by the prefix “C_{x-y}” or “Cₓ—C_{y}—”, wherein x is the minimum and y is the maximum number of carbon atoms in the substituent. Thus, for example, “C₁₋₄alkyl^{”} or “C₁-C₄-alkyl” refers to an alkyl substituent containing from 1 to 4 carbon atoms.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “″effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The term “subject” or “patient” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating,” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. COMPOUND

In one aspect, the present disclosure provides a compound of formula (I′), or a pharmaceutically acceptable salt thereof,

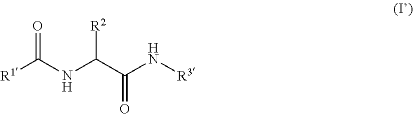

wherein

R¹′ is an aryl or a heteroaryl, wherein R¹′ is optionally substituted with one or more R^{a}′;

R²′ is C₁₋₆alkyl, aryl, heteroaryl, C₁₋₄alkylene-aryl, or C₁₋₄alkylene-heteroaryl, wherein R²′ is optionally substituted with one or more R^{b}′;

R³′ is an aryl or a heteroaryl, wherein R³′ is optionally substituted with one or more R^{c}′;

R^{a}′, R^{b}′, and R^{c}′ at each occurrence are independently halogen, —CN, nitro, N₃, —SO₂NH₂, or —Y—R^{Y};

Y is bond, O, NH, C(O), OC(O), C(O)NH, or S; and R^{Y} is H, alkyl, alkenyl, alkynyl, alkoxy, cycloalkyl, aryl, heteroaryl, or heterocyclyl, wherein R^{Y} is optionally substituted;

provided that the compound is not N-(3-methyl-1-oxo-1-(thiazol-2-ylamino)butan-2-yl)thiophene-2-carboxamide.

In some embodiments, the compound of formula (I′) is enantiomerically enriched. In some embodiments, the compound is compound of formula (I′-a), or a pharmaceutically acceptable salt thereof,

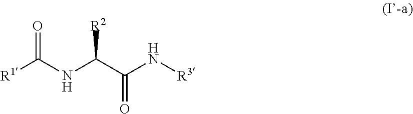

In some embodiments, R¹′ is an optionally substituted 5- to 10-membered heteroaryl, such as an optionally substituted 2-thiophenyl, 3-thiophenyl, 2-indolyl, or 3-indolyl. In some embodiments, R¹′ is unsubstituted. In some embodiments, R¹′ is substituted with one or more —OH, —NH₂, halogen, —CN, nitro, N₃, or —Y—R^{Y}, in which Y is bond, O, NH, C(O), OC(O), C(O)NH, or S; and R^{Y} is alkyl, cycloalkyl, aryl, heteroaryl, or heterocyclyl. In some embodiments, R¹′ is substituted with one or more alkyl, —OH, alkoxy, —NH₂, —NHalkyl, C(O)alkyl, —OC(O)-alkyl, —C(O)O-alkyl, —C(O)NH-alkyl, —NHC(O)-alkyl, —SH, —S-alkyl, halogen, —CN, nitro, or N₃. The alkyl in these substituents may be a C₁₋₁₀alkyl, such as a C₁₋₆alkyl or a C₁₋₄alkyl. In particular embodiments, R¹′ is

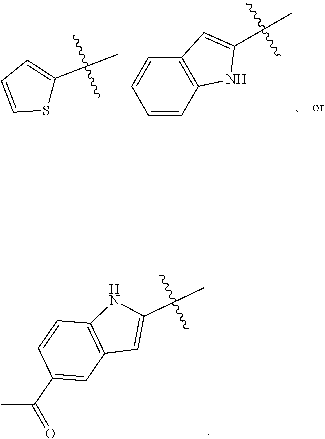 [SMILES: *c1cccs1 ; *c1cc2ccccc2[nH]1 ; *c1cc2cc(C(C)=O)ccc2[nH]1]

In some embodiments, R²′ is an optionally substituted C₁₋₆alkyl or C₁₋₄alkylene-aryl. For example, R²′ may have a structure of a side chain of a natural amino acid, such as hydrophobic (valine, leucine, isoleucine, methionine) or aromatic (phenyalanine, tyrosine, tryptophan) side chains. In some embodiments, R²′ is C₂₋₄alkyl or benzyl. In particular embodiments, R²′ is isopropyl.

In some embodiments, R³′ is an optionally substituted 5- to 10-membered heteroaryl. In some embodiments, R³′ is unsubstituted. In some embodiments, R³′ is substituted with one or more —OH, —NH₂, halogen, —CN, nitro, N₃, or —Y—R^{Y}, in which Y is bond, O, NH, C(O), OC(O), C(O)NH, or S; and R^{Y} is alkyl, cycloalkyl, aryl, heteroaryl, or heterocyclyl. In some embodiments, R³′ is substituted with one or more alkyl, —OH, alkoxy, —NH₂, —NHalkyl, C(O)alkyl, —OC(O)-alkyl, —C(O)O-alkyl, —C(O)NH-alkyl, —NHC(O)-alkyl, —SH, —S-alkyl, halogen, —CN, nitro, or N₃. The alkyl in these substituents may be a C₁₋₁₀alkyl, such as a C₁₋₆alkyl or a C₁₋₄alkyl. In some embodiments, embodiments, R³′ is

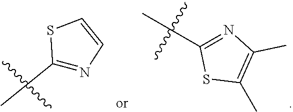

In some embodiments, embodiments, R³′ is

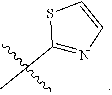 [SMILES: *c1nccs1]

In some embodiments, R¹′ is

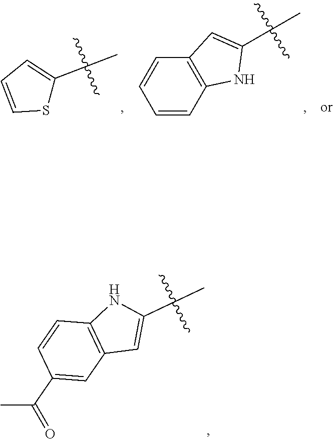 [SMILES: *c1cccs1 ; *c1cc2ccccc2[nH]1 ; *c1cc2cc(C(C)=O)ccc2[nH]1]

R¹′ being optionally substituted with one or more substituents as described herein, R²′ is isopropyl or benzyl, and R³′ is as described herein.

In some embodiments, R¹′ is as described herein, R²′ is isopropyl or benzyl, and R³′ is

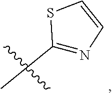 [SMILES: *c1nccs1]

is R³′ being optionally substituted with one or more substituents as described herein.

In some embodiments, R¹′ is

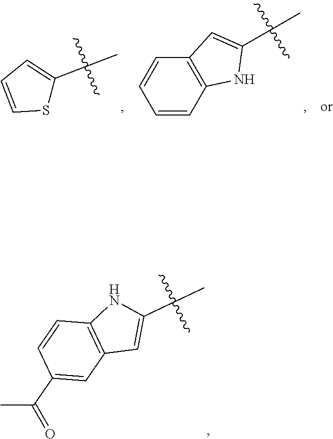 [SMILES: *c1cccs1 ; *c1cc2ccccc2[nH]1 ; *c1cc2cc(C(C)=O)ccc2[nH]1]

R²′ is as described herein, and R³′ is

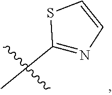 [SMILES: *c1nccs1]

each of R¹′ and R³′ being optionally substituted with one or more substituents as described herein. In particular embodiments, R¹′ is

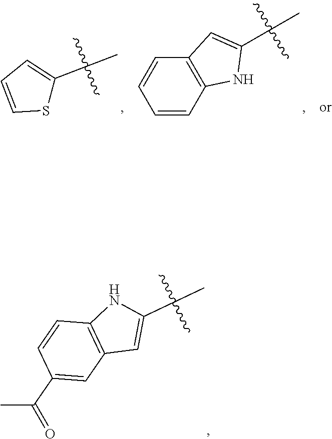 [SMILES: *c1cccs1 ; *c1cc2ccccc2[nH]1 ; *c1cc2cc(C(C)=O)ccc2[nH]1]

R²′ is as described herein, and R³′ is

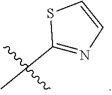 [SMILES: *c1nccs1]

In some embodiments, the compound of formula (I′) is selected from the group consisting of

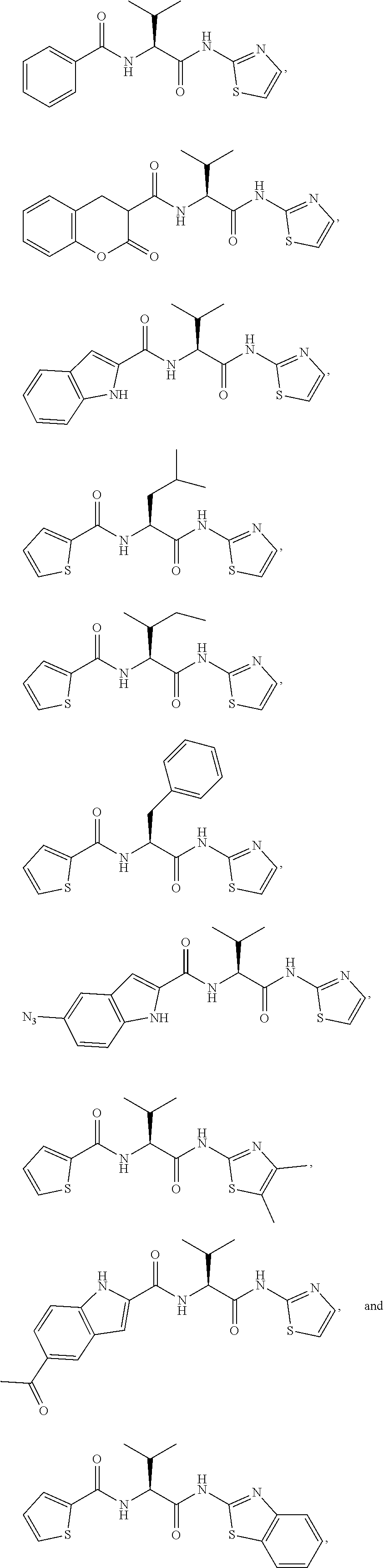

or a pharmaceutically acceptable salt thereof.

In particular embodiments, the compound of formula (I′) is selected from the group consisting of

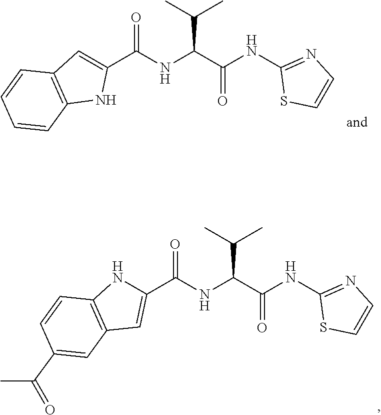 [SMILES: CC(C)[C@H](NC(=O)c1cc2ccccc2[nH]1)C(=O)Nc1nccs1 ; CC(=O)c1ccc2[nH]c(C(=O)N[C@H](C(=O)Nc3nccs3)C(C)C)cc2c1]

or a pharmaceutically acceptable salt thereof.

The present disclosure also provides an enantiomerically enriched (S)—N-(3-methyl-1-oxo-1-(thiazol-2-ylamino)butan-2-yl)thiophene-2-carboxamide

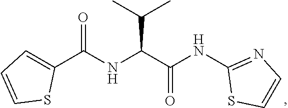 [SMILES: CC(C)[C@H](NC(=O)c1cccs1)C(=O)Nc1nccs1]

or a pharmaceutically acceptable salt thereof. In some embodiments, the compound has an enantiomeric excess (ee) value of at least 90%, such as at least 91%, at least 92%, at least 93%, at least 94%, at least 95%, at least 96%, at least 97%, at least 98%, or at least 99%.

The compounds or compositions disclosed herein may be in the form of a salt, such as a pharmaceutically acceptable salt. As used herein, the term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J Pharmaceutical Sciences, 1977, 66, 1-19, incorporated herein by reference. Pharmaceutically acceptable salts of the present compounds include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N(C₁₋₄alkyl)4 salts. The present disclosure also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed with counterions such as halide, hydroxide, carboxylate, sulfate, phosphate, nitrate, lower alkyl sulfonate and aryl (e.g., phenyl/substituted phenyl) sulfonate.

Compound names are assigned by CHEMDRAW®. The compound may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this disclosure. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods. It should be understood that the compound may possess tautomeric forms, as well as geometric isomers, and that these also constitute an aspect of the present disclosure.

### 3. METHOD

The present disclosure relates to method of inhibiting HSP 70 proteins. Canonical HSP70 proteins contain a nucleotide binding domain (NBD) and a substrate binding domain (SBD) connected by a flexible linker. The NBD is composed of 4 lobes, and the SBD is composed of a beta sheet-rich base and an alpha helix-rich lid. A complex allosteric network exists between these domains where ATP binding to the NBD causes complex rearrangement throughout both domains leading to the opening of the helical lid. Once open, non-native substrate proteins associate thorough the action of an HSP40 cochaperone, and these associations signal the NBD to hydrolyze the bound ATP. Once hydrolyzed, the lid closes on the substrate protein and HSP40 dissociates. Next, a nucleotide exchange factor associates with the NBD and replaces the ADP with ATP. This opens the lid, releases the substrate protein, and starts the cycle over again. As simple as it seems, this cycle of binding and releasing is sufficient to reduce aggregation of non-native polypeptides, promoting their ability to reach their native states. Through this standard cycle and a plethora of cofactors, HSP70s directly play numerous roles in protein folding, disaggregation, proteolytic degradation, organellular translocation, and complex assembly.

The human proteome contains 13 HSP70 family members. Of these, eight are considered canonical. The other five are considered non-canonical due to either lack of a canonical domain or the presence of an extra or non-standard domain. Of the eight canonical HSP70 proteins found in humans, two are restricted to specific organelles. The first is GRP78 (henceforth referred to as HSPA5) which is found in the endoplasmic reticulum (ER), and the second is GRP75 (henceforth HSPA9), found in the mitochondria. The other six isoforms are found primarily in the cytoplasm and can be generally classified by tissue specificity and heat inducibility.

As central regulators of proteostasis, HSP70 proteins are hypothesized targets for anti-cancer and neurodegenerative therapies. In cancer, the role of HSP70 is complicated. It has been shown that most if not all cancers have at least one HSP70 isoform over-expressed due to the increased growth and protein synthesis rate of cancer cells. In addition, cancer cells often produce proteins that contain destabilizing mutations. It has been shown that HSP70 over-expression makes cells more tolerant of mutations as compared to cells with normal levels of HSP70 or high levels of other chaperones. In addition, oncogenic proteins such as XIAP, mutant p53, and Rab1A require HSP70 interactions to be expressed at high levels.

A recent crystallographic study of all the NBDs from human HSP70s shows no unique pockets present in any individual HSP70. Since humans have many more HSP40s than HSP70s it is often thought that HSP70s must somehow be able to recognize different HSP40 proteins. This specificity is a point of divergent function in HSP70 isoforms and has driven the discovery of HSP40 competitive inhibitors. A more overlooked, but not completely unstudied point of divergence within HSP70 isoforms is that of their substrate interactions. While there are no comprehensive reports of HSP70 substrate binding variance, studies examining peptide interaction with a small group of HSP70s show clear differences between DnaK, HSPA8, and HSPA5 with both biochemical assays and phage display. If reported substrate binding affinities are compared across publications in the HSP70 field, trends emerge. For example, the peptide NRLLLTG binds to HSPA5 and DnaK with ˜100 nM affinity, and binds to HSPA1A and HSPA8 with 10 μM affinity. This 100-fold difference is often overlooked because few studies exist directly comparing NRLLLTG binding to multiple isoforms in a single experiment.

According to computational studies, there are 5 sites within HSP70 proteins that are targetable for inhibitor development based on their size, accessibility and hydrophobicity. Two of these sites are ATP-competitive, one is competitive with HSP40, one is allosteric on the substrate binding domain, and one is competitive on the substrate binding domain. Inhibitors have been discovered and characterized for each of these binding sites including VER155008, YK-05, PES, YM01, and the peptide NRLLLTG. Surprisingly, competitive inhibitors of substrate biding such as NRLLLTG are not pursued nearly as often as ATP competitive or HSP40 competitive inhibitors. An assay was recently reported that allows for high throughput screening of inhibitors that disrupt HSPA5-NRLLLTG binding (Ambrose et al., Bioorg. Med. Chem. Lett., 2019, 29 (14), 1689-1693). This assay may be also conducted with the bacterial HSP70 DnaK, but it was unsuccessful with other human HSP70 isoforms due to their lack of affinity for NRLLLTG. Even with this limitation it was shown that hexachlorophene inhibits HSPA5 substrate binding more potently than DnaK substrate binding. The present disclosure expands the fluorescence polarization screen to 70,000 compounds and broaden the substrate association assay to cover more canonical human HSP70 isoforms.

In one aspect, the present disclosure provides a method for inhibiting a heat shock protein 70 (HSP70) comprising contacting the HSP70 with a pharmaceutically effective amount of a compound of formula (I), or a pharmaceutically acceptable salt thereof,

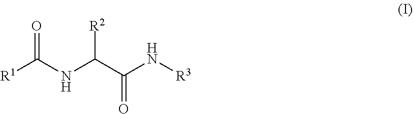

wherein

R¹ is an aryl or a heteroaryl, wherein R¹ is optionally substituted with one or more R^{a};

R² is C₁₋₆alkyl, aryl, heteroaryl, C₁₋₄alkylene-aryl, or C₁₋₄alkylene-heteroaryl, wherein R² is optionally substituted with one or more R^{b};

R³ is an aryl or a heteroaryl, wherein R³ is optionally substituted with one or more R^{c};

R^{a}, R^{b}, and R^{c}at each occurrence are independently halogen, —CN, nitro, N₃, —SO₂NH₂, or —X—R^{X},

X is bond, O, NH, C(O), OC(O), C(O)NH, or S; and

R^{X} is H, alkyl, alkenyl, alkynyl, alkoxy, cycloalkyl, aryl, heteroaryl, or heterocyclyl, wherein R^{X} is optionally substituted.

In some embodiment, the HSP70 comprises at least one isoforms of HSP70. Suitable HSP70 targets include, but are not limited to, HSPA1A, HSPA1B, HSPA1L, HSPA2, HSPA5, HSPA6, HSPA8, HSPA9, and other known isoforms. In some embodiments, the HSP70 comprises HSPA1A, HSPA1B, HSPA1L, HSPA2, HSPA5, HSPA6, HSPA8, HSPA9, or a combination thereof. In some embodiments, the HSP70 comprises HSPA5.

In some embodiments, at least one HSP70 is selectively inhibited by the disclosed compounds, or pharmaceutically acceptable salts thereof. Selectivity may be defined, for example, by comparing the potentency of a disclosed compound in inhibiting various HSP70 isoforms. In particular embodiments, the disclosed compounds, or pharmaceutically acceptable salts thereof, selectively inhibit HSPA5. For example, the compounds may inhibit HSPA5 with a potency that is at least 5-fold, at least 10-fold, at least 20-fold, at least 50-fold, or even 100-fold higher than the potency of the compound to inhibit other HSP70 isoforms.

In some embodiments, the method for inhibiting HSP70 as disclosed herein further comprises contacting the HSP70 with an additional active agent. For example, the additional active agent may be a known p97 inhibitor, such as CB5083.

In another aspect, the present disclosure provides a method for treating a disease characterized by overexpression of a heat shock protein 70 (HSP70) comprising administrating to a subject in need thereof a pharmaceutically effective amount of a compound of formula (I), or a pharmaceutically acceptable salt thereof,

wherein

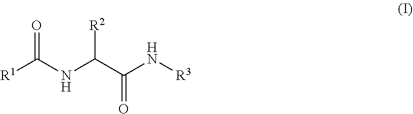

R¹ is an aryl or a heteroaryl, wherein R¹ is optionally substituted with one or more R^{a};

R² is C₁₋₆alkyl, aryl, heteroaryl, C₁₋₄alkylene-aryl, or C₁₋₄alkylene-heteroaryl, wherein R² is optionally substituted with one or more R^{b};

R³ is an aryl or a heteroaryl, wherein R³ is optionally substituted with one or more R^{c};

R^{a}, R^{b}, and R^{c} at each occurrence are independently halogen, —CN, nitro, N₃, —SO₂NH₂, or —X—R^{X},

X is bond, O, NH, C(O), OC(O), C(O)NH, or S; and

R^{X} is H, alkyl, alkenyl, alkynyl, alkoxy, cycloalkyl, aryl, heteroaryl, or heterocyclyl, wherein R^{X} is optionally substituted.

In some embodiments, the disease is cancer. In some embodiments, the cancer is bladder cancer, breast cancer, cervical cancer, colorectal cancer, esophageal cancer, glioblastoma, endometrial cancer, leukemia, liver cancer, lung cancer, mantle cell lymphoma, melanoma, multiple myeloma, oral cancer, ovarian cancer, prostate cancer, pancreatic cancer, renal cancer, stomach cancer, testicular cancer, thyroid cancer, or a combination thereof. In some embodiments, the cancer is breast cancer, colorectal cancer, lung cancer, renal cancer, or a combination thereof

In some embodiments, the method for treating a disease characterized by overexpression of HSP70 as disclosed herein further comprises administrating to the subject an additional active agent. For example, the additional active agent may be a known p97 inhibitor, such as CB5083.

In some embodiments, the compound of formula (I) is enantiomerically enriched. In some embodiments, the compound is compound of formula (I-a), or a pharmaceutically acceptable salt thereof,

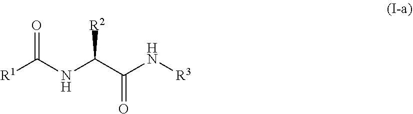

In some embodiments, R¹ is an optionally substituted 5- to 10-membered heteroaryl, such as 2-thiophenyl, 3-thiophenyl, 2-indolyl, or 3-indolyl. In some embodiments, R¹ is unsubstituted. In some embodiments, R¹ is substituted with one or more —OH, —NH₂, halogen, —CN, nitro, N₃, or —X—R^{X}, in which X is bond, O, NH, C(O), OC(O), C(O)NH, or S; and R^{X} is alkyl, cycloalkyl, aryl, heteroaryl, or heterocyclyl. In some embodiments, R¹ is substituted with one or more alkyl, —OH, alkoxy, —NH₂, —NHalkyl, C(O)alkyl, —OC(O)—alkyl, —C(O)O-alkyl, —C(O)NH-alkyl, —NHC(O)-alkyl, —SH, —S-alkyl, halogen, —CN, nitro, or N₃. The alkyl in these substituents may be a C₁₋₁₀alkyl, such as a C₁₋₆alkyl or a C₁₋₄alkyl.

In particular embodiments, R¹ is

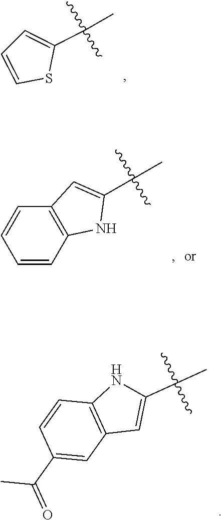 [SMILES: *c1cccs1 ; *c1cc2ccccc2[nH]1 ; *c1cc2cc(C(C)=O)ccc2[nH]1]

In some embodiments, R² is an optionally substituted C₁₋₆alkyl or C₁₋₄alkylene-aryl. For example, R² may have a structure of a side chain of a natural amino acid, such as hydrophobic (valine, leucine, isoleucine, methionine) or aromatic (phenyalanine, tyrosine, tryptophan) side chains. In some embodiments, R² is C₂₋₄alkyl or benzyl. In particular embodiments, R² is isopropyl.

In some embodiments, R³ is an optionally substituted 5- to 10-membered heteroaryl. In some embodiments, R³ is unsubstituted. In some embodiments, R³ is substituted with one or more —OH, —NH₂, halogen, —CN, nitro, N₃, or —X—R^{X}, in which X is bond, O, NH, C(O), OC(O), C(O)NH, or S; and R^{X} is alkyl, cycloalkyl, aryl, heteroaryl, or heterocyclyl. In some embodiments, R³ is substituted with one or more alkyl, —OH, alkoxy, —NH₂, —NHalkyl, C(O)alkyl, —OC(O)-alkyl, —C(O)O-alkyl, —C(O)NH—alkyl, —NHC(O)-alkyl, —SH, —S-alkyl, halogen, —CN, nitro, or N₃. The alkyl in these substituents may be a C₁₋₁₀alkyl, such as a C₁₋₆alkyl or a C₁₋₄alkyl. In some embodiments, R³ is

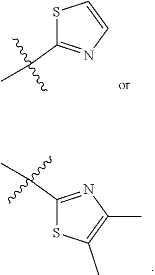

In some embodiments, R³ is

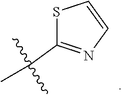 [SMILES: *c1nccs1]

In some embodiments, R¹ is

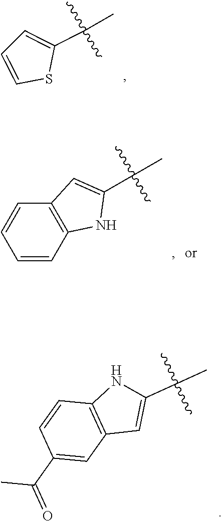 [SMILES: *c1cccs1 ; *c1cc2ccccc2[nH]1 ; *c1cc2cc(C(C)=O)ccc2[nH]1]

R¹ being optionally substituted with one or more substituents as described herein, R² is isopropyl or benzyl, and R³ is as described herein.

In some embodiments, R¹ is as described herein, R² is isopropyl or benzyl, and R³ is

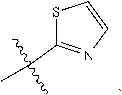 [SMILES: *c1nccs1]

R³′ being optionally substituted with one or more substituents as described herein.

In some embodiments, R¹ is

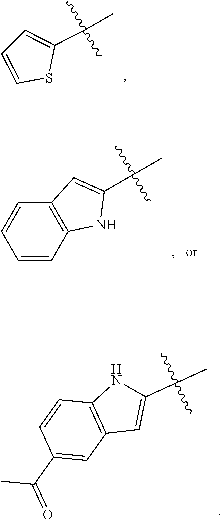 [SMILES: *c1cccs1 ; *c1cc2ccccc2[nH]1 ; *c1cc2cc(C(C)=O)ccc2[nH]1]

R² is as described herein, and R³ is

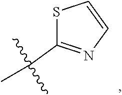 [SMILES: *c1nccs1]

each of R¹ and R³ being optionally substituted with one or more substituents as described herein. In particular embodiments, R¹ is

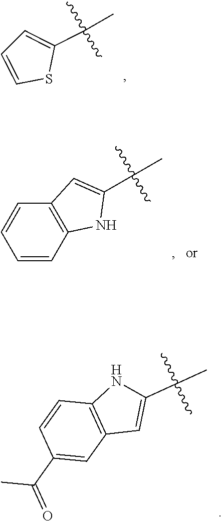 [SMILES: *c1cccs1 ; *c1cc2ccccc2[nH]1 ; *c1cc2cc(C(C)=O)ccc2[nH]1]

r² is as described herein, and R³ is

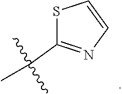 [SMILES: *c1nccs1]

In some embodiments, the compound of formula (I) is selected from the group consisting of

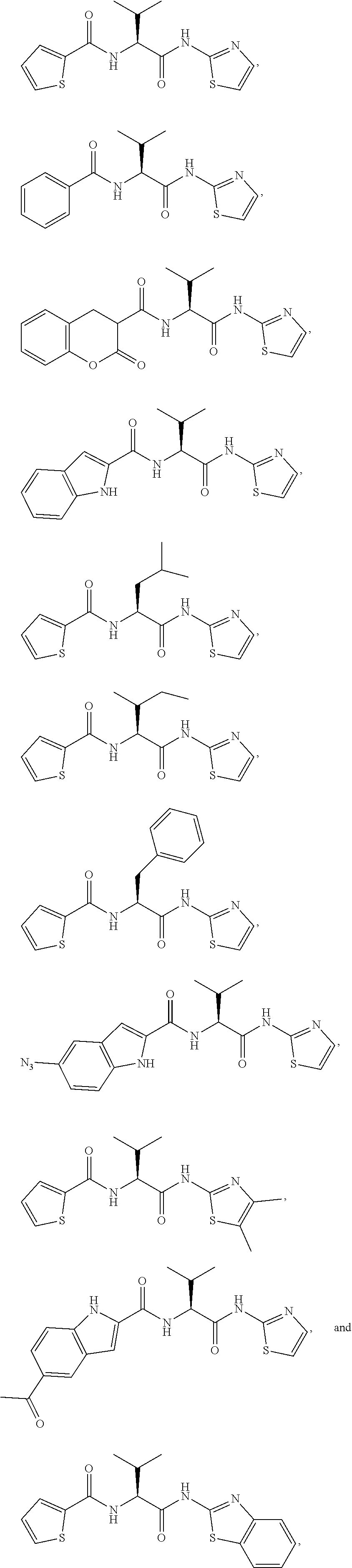

or a pharmaceutically acceptable salt thereof.

In particular embodiments, the compound of formula (I) is selected from the group consisting of

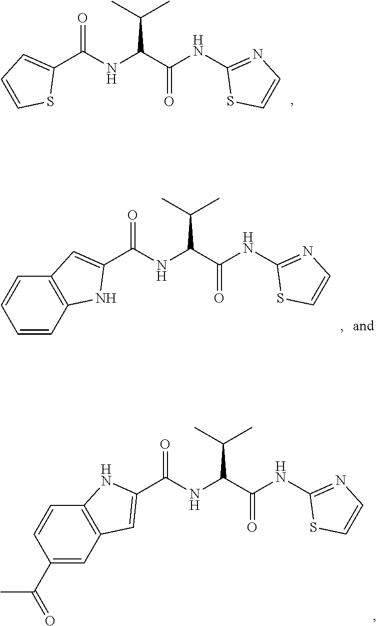

or a pharmaceutically acceptable salt thereof.

Administration

The present compounds or compositions may be administered to a subjects by a variety of known routes, including without limitation oral, inhalation, intravenous, intramuscular, topical, subcutaneous, systemic, and/or intraperitoneal administration.

The amount of the present compounds, or a pharmaceutically acceptable salts thereof, for use in treatment may vary with the particular compound or salt selected, the route of administration, the disease or condition being treated, and the age and condition of the subject being treated. In cases of administration of a pharmaceutically acceptable salt, dosages may be calculated as the free base. In certain situations the disclosed compounds may be administered in amounts that exceed the dosage ranges described herein in order to effectively and aggressively treat particularly aggressive diseases or conditions.

In some embodiments, the compounds, or pharmaceutically acceptable salts thereof, or pharmaceutical compositions as disclosed herein may be administered by inhalation, oral administration, or intravenous administration. In general, however, a suitable dose will often be in the range of from about 0.01 mg/kg to about 100 mg/kg, such as from about 0.05 mg/kg to about 10 mg/kg. For example, a suitable dose may be in the range from about 0.10 mg/kg to about 7.5 mg/kg of body weight per day, such as about 0.10 mg/kg to about 0.50 mg/kg of body weight of the recipient per day, about 0.10 mg/kg to about 1.0 mg/kg of body weight of the recipient per day, about 0.15 mg/kg to about 5.0 mg/kg of body weight of the recipient per day, about 0.2 mg/kg to 4.0 mg/kg of body weight of the recipient per day. The compound may be administered in unit dosage form; for example, containing 1 to 100 mg, 10 to 100 mg or 5 to 50 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

Suitable in vivo dosage to be administered and the particular mode of administration may vary depending upon the age, weight, the severity of the affliction, and mammalian species treated, the particular compounds employed, and the specific use for which these compounds are employed. The determination of effective dosage levels to achieve the desired result may be accomplished by known methods, for example, human clinical trials, in vivo studies and in vitro studies. For example, the effective dosages of compounds disclosed herein, or pharmaceutically acceptable salts thereof, may be determined by comparing their in vitro activity, and in vivo activity in animal models. Such comparison may be done by comparison against an established drug.

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, FIPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

Compounds, salts, and compositions disclosed herein may be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

The compositions described herein may be administered with additional compositions to prolong stability, delivery, and/or activity of the compositions, or combined with additional therapeutic agents, or provided before or after the administration of additional therapeutic agents. Combination therapy includes administration of a single pharmaceutical dosage formulation containing one or more of the compounds described herein and one or more additional pharmaceutical agents, as well as administration of the compounds and each additional pharmaceutical agent, in its own separate pharmaceutical dosage formulation.

### 4. PHARMACEUTICAL COMPOSITIONS

In another aspect, the present disclosure provides a pharmaceutical composition comprising a compound as disclosed herein, or a pharmaceutically acceptable salt there, and a pharmaceutically acceptable carrier.

The present pharmaceutical compositions may be manufactured by processes known in the art, e.g., by means of conventional mixing, dissolving, granulating, dragee-making, levigating, emulsifying, encapsulating, entrapping or lyophilizing processes.

As described herein, the pharmaceutically acceptable carrier includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Various carriers used in formulating pharmaceutically acceptable compositions and techniques for the preparation thereof are known in the art (e.g., Remington's Pharmaceutical Sciences, Sixteenth Edition, E. W. Martin (Mack Publishing Co., Easton, Pa., 1980)).

Some examples of materials which can serve as pharmaceutically acceptable carriers include, but are not limited to, ion exchangers, alumina, aluminum stearate, lecithin, serum proteins (such as human serum albumin), buffer substances (such as phosphates), glycine, sorbic acid, or potassium sorbate, partial glyceride mixtures of saturated vegetable fatty acids, water, salts or electrolytes (such as protamine sulfate, disodium hydrogen phosphate, potassium hydrogen phosphate, sodium chloride, zinc salts), colloidal silica, magnesium tri silicate, polyvinyl pyrrolidone, polyacrylates, waxes, polyethylenepolyoxypropylene-block polymers, wool fat, sugars (such as lactose, glucose, and sucrose), starches (such as com starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil, soybean oil), glycols (such a propylene glycol or polyethylene glycol), esters (such as ethyl oleate and ethyl laurate), agar, non-toxic compatible lubricants (such as sodium lauryl sulfate and magnesium stearate), coloring agents, releasing agents, coating agents, emulsifying agents, sweetening, flavorant, perfuming agents, preservatives, antioxidants can also be present in the composition, according to the judgment of the formulator.

Liquid dosage forms for oral administration include, but are not limited to, pharmaceutically acceptable emulsions, microemulsions, solutions, suspensions, syrups and elixirs. Solid dosage forms for oral administration include, but are not limited to, capsules, tablets, pills, powders, cement, putty, and granules. Dosage forms for topical or transdermal administration of the present compounds include, but are not limited to, ointments, pastes, creams, lotions, gels, powders, solutions, sprays, inhalants or patches.

### 5. EXAMPLES

General synthesis for diamide compounds. Representative compounds were prepared according to Scheme 1.

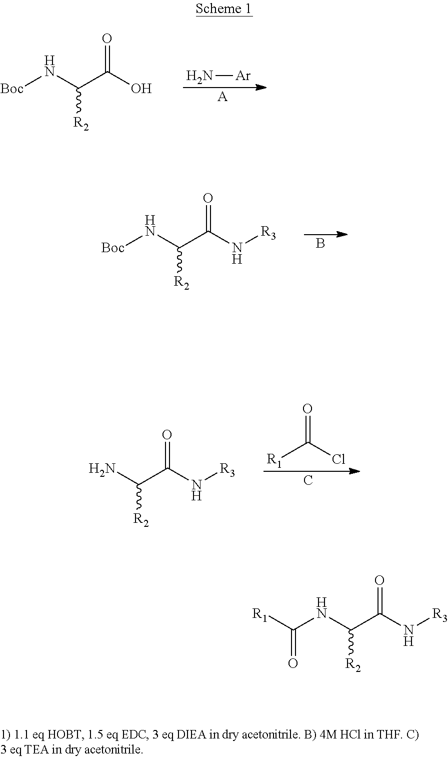

Boc-amino acid amides. N-Boc-amino acid (2 mmol) is dissolved in 8 mLs of acetonitrile (DMF for phenylalanine and oxazole amine) and 2.2 mmol of HOAT and 3 mmol of EDC are added and the mixture was stirred for 30 minutes. After activation of the acid, 2.5 mmol of aromatic amine and 6 mmol of DIEA were added. The reaction proceeded overnight and was separated between 100 mM aqueous ascorbic acid and EtOAc. The organic phase was then washed 2× with 100 mM aqueous ascorbic acid, 3× with saturated sodium bicarbonate, 2× with brine, and dried over Na₂SO₄. The desired product was then purified by flash chromatography (10-30% EtOAc in hexanes).

Boc deprotection. The desired Boc-amino acid amide (1 mmole) is dissolved in 10 mLs of 4M HCl in THF and stirred for 16 hr at room temperature. The solvent was removed on a rotary evaporator and the product was recrystallized from MeOH and DCM and collected by filtration.

Acyl chloride synthesis. Aromatic carboxylic acid (10 mmole) is dissolved in 10 mLs of thionyl chloride and stirred at room temperature for 16 hours. The thionyl chloride is removed by vacuum distillation below 35° C. and the crude product is used without further purification.

Reaction of acyl chloride with deprotected amino acid amide. Deprotected amino acid amide (1 mmol) was resuspended in 2 mLs of DMF and 5 mmol TEA was added. To this mixture, was added 3 mmol of the desired acyl chloride and TEA was added in 100 μL portions if needed until the pH of the mixture was ˜10. The mixture was stirred overnight at which point the mixture was diluted in aq bicarbonate extracted 3× with EtOAc. The combined organic phases were washed 3× with saturated sodium bicarbonate, 2× with brine, and dried over Na₂SO₄. After removal of the solvent, the product was purified using flash chromatography (50% EtOAc in hexanes) and recrystallized from Et₂O if necessary.

Compound 1, N-(3-methyl-1-oxo-1-(thiazol-2-ylamino)butan-2-yl)thiophene-2-carboxamide, was purchased from ChemDIV Inc. (San Diego, Calif.).

Protein Production

HSP70 constructs were expressed from pSpeedET vectors with a TEV protease cleavable N terminal his tag. Codon plus E. coli (Invitrogen) were transformed with the respective plasmid and grown on agar plates containing 35 μg/mL chloramphenicol and 50 μg/mL kanamycin. Colonies were washed into 2×YT media in baffled 2 L flasks and grown at 37° C. to an OD of 0.6. At this point, the bacteria were transferred to a room temperature shaking incubator for 1 hr before IPTG was added to a final concentration of 500 μM. The bacteria were grown for 16 hr and harvested by centrifugation. The pellets were resuspended in HKM buffer (50 mM HEPES pH 7.4, 150 mM KCl, 10 mM MgCl₂, 2 mM BME) and lysed by repeated passage through a microfluidizer (microfluidics corporation) at 12,000 PSI. The lysate was clarified by centrifugation and incubated with cobalt talon agarose (gold biotech) for 1 hr. The slurry was then applied to a gravity column, washed with 20 CV HKM buffer. The protein of interest was eluted with HKM buffer containing 200 mM imidazole. TEV protease was added to cleave the Hisx6 tag for 16 hr at 4° C. During this time, the eluted protein was extensively dialyzed against HKM buffer. Once cleavage was complete, the TEV protease was recaptured using cobalt resin and the protein was aliquoted and flash frozen in liquid nitrogen.

FP Assays

FP assays were conducted in black 384-well low volume plates (corning) with 10 nM of indicated fluorescent peptide (ABclonal) or 22 in assay buffer (50 mM HEPES pH 7.4, 100 mM KCl, 10 mM MgCl₂. Polarization was measured using the ID5 microplate reader (molecular devices) with an excitation of 485 nm and emission of 535 nm. For Kd determination, respective HSP70s were serial diluted and the data were fit to a one site binding model in PRISM 6.07 (GraphPad). For compound measurements, HSP70 was added at a final concentration of 1 μM and % inhibition was calculated using unlabeled peptide as a positive control and DMSO as a negative control.

PAMPA Assay

PAMPA assays were conducted using the gentlest precoated 96 well PAMPA plates (corning). Compounds were added in 300 μL 1×PBS to the donor plate before the acceptor plate containing 200 μL of 1×PBS was added. The compounds equilibrated for 4 hr before the plates were separated and the contents transferred to HPLC vials. The concentration on each side of the membrane was calculated using reference samples. Permeability in units of cm/s was calculated using Equation 1.

$$P = \frac{- L N ( 1 - \frac{Ca}{Ceq} )}{A * ( \frac{1}{V d} + \frac{1}{V a} )} * t \qquad ( 1 )$$

In Equation 1, Ca is the concentration in the acceptor well at the end of the assay, A is the area of the membrane in cm² (0.3 in this case), Vd is the volume of the donor well in mL, Va is the volume of the acceptor well in mL, t is the time of the assay in seconds, and Ceq is the concentration at equilibrium accounting for compound sticking to the membrane. Ceq was calculated using Equation 2 (where Cd is the concentration remaining in the donor well at the end of the assay).

$$C e q = \frac{[ C d * V d + C a * V a ]}{[ V d + V a ]} \qquad ( 2 )$$

Serum Binding Assay

Serum binding was measured using human serum and the commercially available probes dansylamide and BD140 (TCI chemicals). Both probes exhibit turn-on fluorescence upon binding to HSA and bind to distinct sites on the protein. Compounds were incubated at 50 μM in the wells of a 96 well black plate (corning). Each sample contained 20 μM HSA in PBS at pH 7.4 and either 10 μM dansylamide or 3 μM BD140. Warfarin was used as a positive control for BD140 and ibuprofen (Santa Cruz Biotechnology) as a positive control for dansylamide. DMSO was used as a negative control. After a 30-minute incubation at room temperature, probe binding was evaluated using a universal excitation of 365 nm and an emission of 480 nm for dansylamide and 590 nm for BD140.

Microsome Stability

To evaluate the stability of the compounds, they were incubated at a concentration of 50 μM and a volume of 100 μL with 0.2 mg/ml human liver microsomes (Xenotech). The buffer used was a 1×PBS buffer containing 3 mM MgCl₂, 1 mM EDTA, and an NADPH regeneration system consisting of 1 mM NADP, 5 mM glucose-6-phosphate (G-6-P), and 1 unit/ml G-6-P dehydrogenase. Compounds were incubated for incubated for 4 hr at 37° C. and the reaction was quenched by the addition of 900 μL of acetonitrile. Concentration remaining was then calculated using calibrated LC-MS. Verapamil was used as a positive control.

CACO-Permeability

Caco-2 cells (ATCC HTB-37) obtained from University of Arizona Cancer Center Cell Culture Services, maintained in DMEM (Corning 10013CV), Penicillin-Streptomycin (Thermo 10378016), and 10% FBS (Thermo A3160401), were plated at a density of 2×105 wells into each insert (1 μM pore size) of a Corning Biocoat HTS Caco-2 Assay System 24-well plate (Corning 354801). Monolayer quality was measured by verification of retention of lucifer yellow from either side of membrane at 72 hours post differentiation of Caco-2 cells. In 300 μL transport buffer (pH 6.8 or 7.5), compound of interest was dissolved to 10 μM and added to insert (apical side). Next, 1000 μL of transport buffer alone was added to plate well (basal side). System was placed in cell culture incubator maintained at 37° C. with 5% CO₂ and samples from basal side were obtained after 90 minutes. Experiment was performed testing both apical-to-basal and basal-to-apical transport of compound of interest to determine apparent permeability. Internal standards included propranolol (Sigma P0884) and verapamil (Sigma V4629). Concentration of compound was measured using calibrated HPLC UV traces.

Cell Culture and Thiazolyl Blue Tetrazolium Bromide (MTT) Viability Assay

HCT116, BEAS-2B, CAKI, SW-620, A549, 231T, and SW620 cells were maintained as recommended by the American Type Culture Collection. All media was supplemented with 10 units/ml of penicillin and streptomycin. Cell viability was measured using a standard MTT assay protocol (abcam) and Thiazolyl Blue Tetrazolium Bromide (gold biotechnology). Cells were seeded at 70% confluency in 96 well cell culture plates (greiner). After overnight incubation, compounds were added at indicated concentrations with each well containing 0.5% DMSO. Cells were incubated for 72 hours before 50 uL of 5 mg/ml MTT in PBS was added to each well. Cells were incubated for 3 hours with MTT before the media was carefully removed. MTT crystals were then resuspended in 4 mM HCl and 0.1% tween-20 in isopropanol. The absorbance was then read at 580 nm on an ID5 microplate reader (molecular devices). Cell growth inhibition was calculated by diving a blank subtracted sample from blank subtracted cells treated with DMSO. Cell growth IC₅₀ was calculated using PRISM 6.07 software (GraphPad). ZIP synergy was calculated using the Bioconductor synergy finder package in R.

### Example 1

### HSP 70 Inhibitors

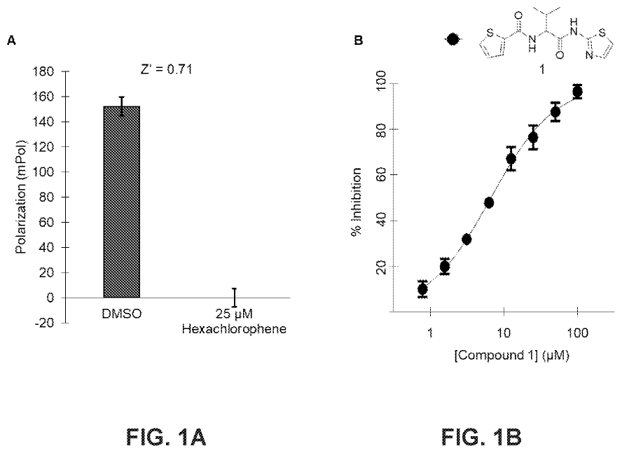

In an effort to find a drug-like and selective inhibitor of HSPA5, a high throughput screen of 70,000 compounds was conducted using our previously published peptide binding fluorescence polarization assay using hexachlorophene as a positive control. From this screen compound 1 was identified as a promising hit due to its synthetic accessibility, reproducibility, and its relatively potent IC₅₀ of 7.5±1.2 μM (FIGS. 1A and 1B).

The assay revealed compounds that prevent the formation of the HSPA5-NRLLLTG complex. It was hypothesized that compound 1 binds to the peptide binding site of HSPA5. Given that the core of 1 is valine, the impact of chirality on the activity of 1 was investigated. By using standard amide coupling procedures both the S (compound 2) and the R (compound 3) enantiomers of 1 were synthesized.

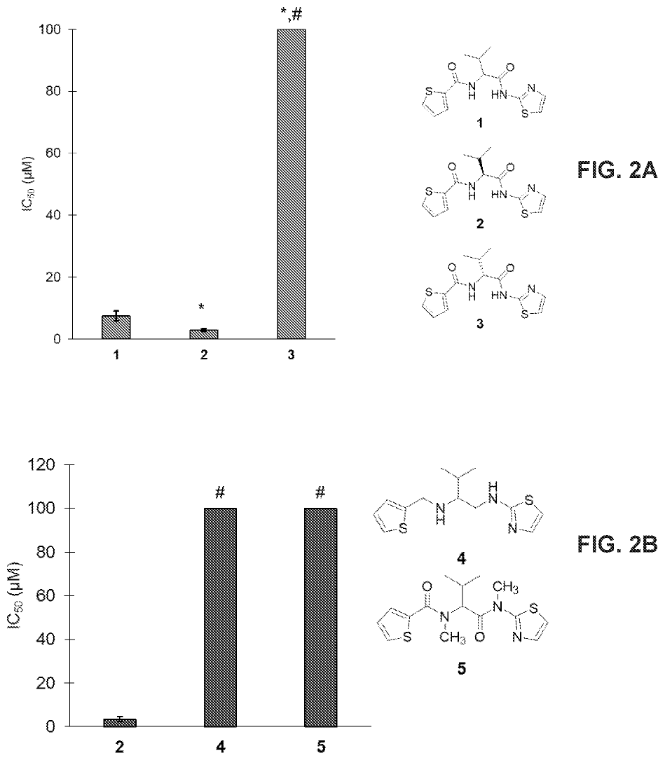

Dose dependent inhibition revealed that the S enantiomer 2 is about twice as potent as 1 (3.5±0.75 μM vs 7.5±1.2 μM) while the R enantiomer 3 did not display detectable inhibition up to 100 μM (FIG. 2A). This observation is consistent with the hypothesis that 1 binds to the substrate binding site of HSPA5 and that native substrates are natural peptides. Further, the effect of the amide bonds in the activity of 1 was studied. Given that HSP70s bind to a varied host of substrate peptides, it was hypothesized that the peptide binding site would rely on the amide backbone of the peptide for binding. To this end, the amide bonds were reduced to provide secondary amines (4) and methylated the amide (5). Neither of compounds 4 and 5 displayed detectable activity up to 100 μM (FIG. 2B). These results indicated the importance of the amide bonds in binding of 1, which supports the hypothesis that this molecule binds to the peptide binding site of HSPA5.

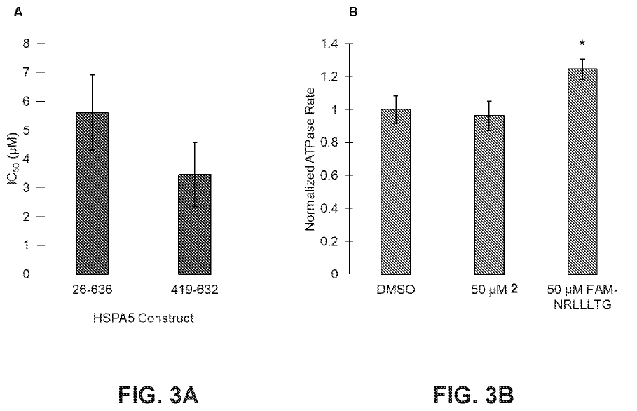

To further investigate the binding site of these molecules, the isolated substrate binding domain of HSPA5 was purified. No significant difference in the activity of 2 was observed with either the full-length protein (26-636) or the isolated substrate binding domain (419-632) (FIG. 3A). The effect of 2 on the ATPase activity of HSPA5 was also investigated, as it has been reported that substrate peptides can accelerate the ATPase rate of HSPA5. No significant effect on the ATPase rate of HSPA5 was observed. However, a subtle but statistically significant acceleration was indeed apparent when the protein was exposed to the peptide substrate used in the peptide binding assay (FIG. 3B). This indicates that although 2 may bind to the peptide binding site, it does not affect the allosteric communication of HSPA5 in the same manner as a peptide substrate.

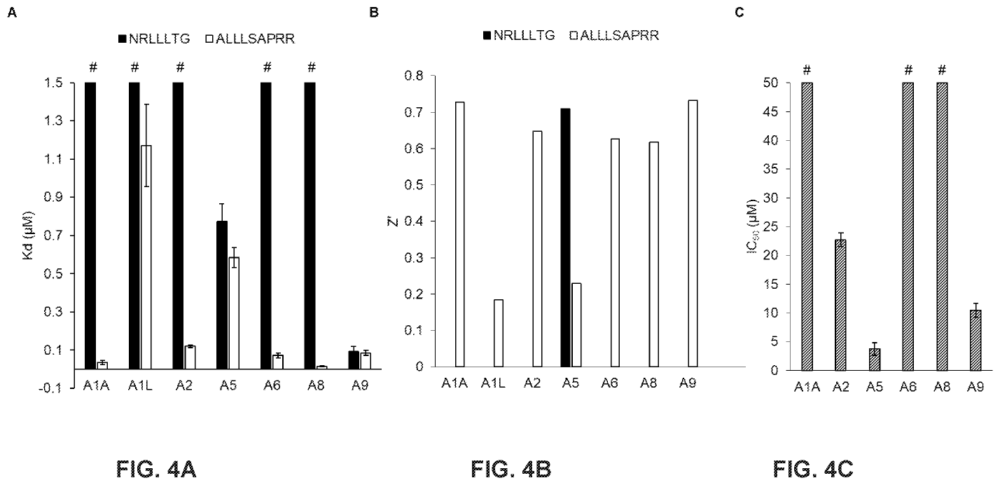

The capacity of compound 2 to inhibit other HSP70s was characterized. Fluorescent peptide (FITC-NRLLLTG) did not bind with high enough affinity to the other human HSP70 substrate binding domains to use it as an FP probe (FIG. 4A). Although somewhat surprising, this is consistent with literature reports that the NRLLLTG binds with much higher affinity to DnaK and HSPA5 than to the proteins encoded by the HSPA1A or HSPA8 genes. There are other fluorescent peptides that have been shown to bind to human HSP70 isoforms with high affinity such as FAM-RENLRIALRY (HLA peptide) or FAM-ALLLSAPRR (P1). When subjected to the canonical human HSP70 isoforms, P1 bound with high affinity to all isoforms except HSPA1L, while the FITC-NR peptide only bound tightly to HSPA5 and HSPA9 (FIG. 4A). With P1, acceptable Z′ factors for all tested HSP70 SBDs at 1 μM were obtained except for HSPA1L (FIG. 4B). Dose dependent inhibition of peptide biding to the respective HSP70 isoforms was measured with 2 revealing high selectivity for HSPA5 and HSPA9 as compared to the other human HSP70 isoforms (FIG.

4C).

Additional molecules with varied aromatic rings at position R₁ (amine terminus of amino acid, Table 1) were prepared and significant affinity was observed by replacing the thiophene with and indole (2 vs 8). Compound 8 also gained selectivity compared to compound 2 as 2 is 3-fold selective between HSPA5 and HSPA9 and 10-fold selective between HSPA5 and HSPA2. On the other hand, 8 is 7-fold selective for HSPA5 compared to HSPA9 and >20-fold selective for HSPA5 compared to HSPA2 (Table 1). In general, modification of this position was well tolerated with only 6 losing potency as compared to 2. Coumarin substitution (7) was slightly less potent, and drastically less selective than either thiophene or indole derivatives (Table 1).

TABLE 1

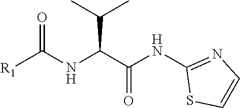

| | | A1A IC₅₀ | A2 IC₅₀ | A5 IC₅₀ | A6 IC₅₀ | A8 IC₅₀ | A9 IC₅₀ |
|---|---|---|---|---|---|---|---|
| # | R₁ | (μM) | (μM) | (μM) | (μM) | (μM) | (μM) |
| 2 | 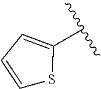 [SMILES: *c1cccs1] | >50 | 22.7 +/− 1.8 | 2.9 +/− 0.5 | >50 | >50 | 10.1 +/− 1.2 |
| 6 | 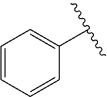 [SMILES: *c1ccccc1] | >50 | >50 | 11.0 +/− 1.2 | >50 | >50 | 20.5 +/− 1.1 |
| 7 | 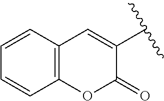 [SMILES: *c1cc2ccccc2oc1=O] | 25.2 +/− 1.1 | 25.2 +/− 1.4 | 6.6 +/− 1.2 | >50 | >50 | 13.6 +/− 1.3 |
| 8 | 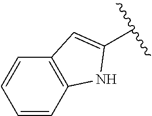 [SMILES: *c1cc2ccccc2[nH]1] | >50 | 13.9 +/− 1.2 | 0.59 +/− 0.06 | >50 | 43.5 +/− 6.4 | 4.3 +/− 1.3 |

Next, the core amino acid of 2 was varied (Table 2, R₂). In some cases, hydrophobic amino acids were maintained, given that solved structures of peptides bound to HSP70 always have 1 hydrophobic amino acid buried in a hydrophobic pocket in the beta sheet rich portion of the SBD. Additionally, efforts toward finding HSP70 binding consensus peptide sequences have pointed to groups of 7 amino acids with a hydrophobic core and basic flanking residues. The present data indicated that changing the amino acid from valine to alanine results in a complete loss of inhibition (9). Isoleucine and leucine are slightly improved in HSPA5 inhibition (10 and 12) but are slightly more promiscuous than valine. Phenylalanine (11) is interesting in that its effect on HSPA5 inhibition is very subtle, but it changes the panel of inhibition significantly. Despite having less affinity for HSPA5, 12 is the most potent binder of HSPA2. Valine was chosen for further development in this study, but in general, variation of this position has potential in developing selective molecules for other HSP70 isoforms.

TABLE 2

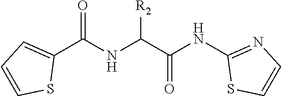

| | | A1A IC₅₀ | A2 IC₅₀ | A5 IC₅₀ | A6 IC₅₀ | A8 IC₅₀ | A9 IC₅₀ |
|---|---|---|---|---|---|---|---|
| # | R₂ | (μM) | (μM) | (μM) | (μM) | (μM) | (μM) |
| 2 | 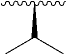 | >50 | 22.7 +/− 1.8 | 2.9 +/− 0.5 | >50 | >50 | 10.1 +/− 1.2 |
| 9 | 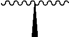 | >50 | >50 | >50 | >50 | >50 | >50 |
| 10 | 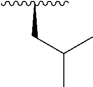 | 49.0 +/− 1.2 | 15.2 +/− 1.7 | 1.2 +/− 0.2 | >50 | >50 | 7.1 +/− 1.8 |
| 11 | 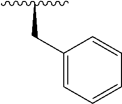 | 47.3 +/−1.8 | 6.8 +/− 1.5 | 10.4 +/− 1.1 | >50 | >50 | 14.3 +/− 1.3 |
| 12 | 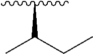 | >50 | 6.0 +/− 1.1 | 1.7 +/− 0.19 | >50 | 12.7 +/− 1.2 | 23.8 +/− 1.1 |

Molecules with varying groups at the carboxy terminus of the amino acid were made (Table 3, R₃). The results demonstrate that this position appears to be the least tolerant as even subtle replacement of thiazaole with oxazole or 3,4-thiadizaole results in a drastic loss of activity. Various aliphatic esters (18-21) placed at this position resulted in a drastic loss of activity.

TABLE 3

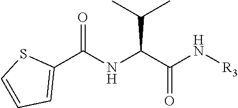

| | | A1A IC₅₀ | A2 IC₅₀ | A5 IC₅₀ | A6 IC₅₀ | A8 IC₅₀ | A9 IC₅₀ |
|---|---|---|---|---|---|---|---|
| # | R₃ | (μM) | (μM) | (μM) | (μM) | (μM) | (μM) |
| 2 | 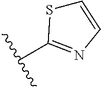 [SMILES: *c1nccs1] | >50 | 22.7 +/− 1.8 | 2.9 +/− 0.5 | >50 | >50 | 10.1 +/− 1.2 |
| 13 | 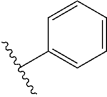 [SMILES: *c1ccccc1] | N/A | N/A | >50 | N/A | N/A | N/A |
| 14 | 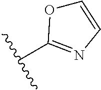 [SMILES: *c1ncco1] | N/A | N/A | >50 | N/A | N/A | N/A |
| 15 | 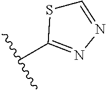 [SMILES: *c1nncs1] | N/A | N/A | >50 | N/A | N/A | N/A |
| 16 | 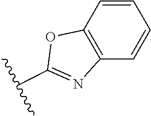 [SMILES: *c1nc2ccccc2o1] | N/A | N/A | >50 | N/A | N/A | N/A |
| 17 | OH | N/A | N/A | >50 | N/A | N/A | N/A |
| 18 | OMe | N/A | N/A | >50 | N/A | N/A | N/A |
| 19 | OEt | N/A | N/A | >50 | N/A | N/A | N/A |
| 20 | OPr | N/A | N/A | >50 | N/A | N/A | N/A |
| 21 | OBu | N/A | N/A | >50 | N/A | N/A | N/A |

(S)—N-(3-methyl-1-oxo-1-(thiazol-2-ylamino)butan-2-yl)thiophene-2-carboxamide (2). 1H NMR (400 MHz, Chloroform-d) δ 1.05 (dd, J=3.4, 6.5 Hz, 6H), 2.30 (h, J=7.0 Hz, 1H), 5.03 (t, 1H), 7.03-7.16 (m, 2H), 7.20 (d, J=8.2 Hz, 2H), 7.51 (d, J=4.8 Hz, 1H), 7.63 (d, J=3.1 Hz, 1H), 7.82 (s, 1H), 11.15 (broad, 1H). [α]²⁵ (acetone): 1.66.

(R)-N-(3-methyl-1-oxo-1-(thiazol -2-ylamino)butan-2-yl)thiophene-2-carboxamide (3). 1H NMR (400 MHz, Chloroform-d) δ 1.05 (dd, J=3.4, 6.5 Hz, 6H), 2.30 (h, J=7.0 Hz 1H), 5.03 (t, 1H), 7.03-7.16 (m, 2H), 7.20 (d, J=8.2 Hz, 2H), 7.51 (d, J=4.8 Hz, 1H), 7.63 (d, J=3.1 Hz, 1H), 7.82 (s, 1H), 11.15 (broad, 1H). [α]²⁵ (acetone): 2.22.

3-methyl-N1-(thiazol -2-yl)-N2-(thiophen-2-ylmethyl)butane-1,2-diamine (4). Compound 2 (0.2 mmol) was dissolved in 4 mls of dry THF and 3eq of LiAlH was slowly added on ice. The reaction proceeded overnight at room temperature before it was quenched with ice cold aqueous ammonium chloride. This mixture was extracted 3× with EtOAc and the combined organic layers were dried with Na₂SO₄. The desired compound was purified using silica chromatography (100% EtOAc). 1H NMR (400 MHz, Chloroform-d) δ 1.01 (dd, J=6.8, 19.2 Hz, 6H), 2.05 (h, J=6.9 Hz, 1H), 2.72-2.85 (m, 1H), 3.24 (dd, J=7.7, 12.8 Hz, 1H), 3.47 (dd, J=4.1, 12.8 Hz, 1H), 4.02-4.25 (m, 2H), 6.48 (d, J=3.7 Hz, 1H), 6.97 (d, J=4.3 Hz, 2H), 7.12 (d, J=3.7 Hz, 1H), 7.25 (dd, J=2.0, 4.3 Hz, 1H).

N-methyl-N-(3-methyl-1-(methyl(thiazol-2-yl)amino)-1-oxobutan-2-yl)thiophene carboxamide (5). Compound 2 (0.2 mmol was dissolved in 4 mls of dry THF and 3eq of NaH was slowly added on ice, followed by 6eq MeI. The reaction proceeded overnight at room temperature before it was quenched with ice cold aqueous ammonium chloride. This mixture was extracted 3× with EtOAc and the combined organic layers were dried with Na₂SO₄. The desired compound was purified using silica chromatography (50% EtOAc). 1H NMR (400 MHz, Chloroform-d) δ 0.72-1.13 (dd, 6H), 2.66 (h, J=6.9 Hz 1H), 3.18 (d, J=85.9 Hz, 3H), 3.79 (s, 3H), 4.60 (s, 1H), 6.72 (d, J=4.7 Hz, 1H), 6.96-7.17 (m, 2H), 7.44 (dd, J=1.1, 5.0 Hz, 1H), 7.85 (d, J=4.1 Hz, 1H).

(S)—N-(3-methyl-1-oxo-1-(thiazol-2-ylamino)butan-2-yl)benzamide (6). 1H NMR (400 MHz, Chloroform-d) δ 1.06 (dd, J=4.5, 6.8 Hz, 6H), 2.40 (h, J=6.9 Hz, 1H), 5.07 (dd, J=6.6, 8.8 Hz, 1H), 7.07 (d, J=3.6 Hz, 1H), 7.13 (d, J=8.8 Hz, 1H), 7.38-7.60 (m, 4H), 7.76 (d, J=3.6 Hz, 1H), 7.82-7.91 (m, 1H), 12.70 (s, 1H).

(S)—N-(3-methyl -1-oxo-1-(thiazol-2-ylamino)butan-2-yl)-2-oxo-2H-chromene-3-carboxamide (7). 1H NMR (400 MHz, Chloroform-d) δ 1.14 (d, J=6.8 Hz, 6H), 2.50 (h, J=6.7 Hz, 1H), 4.95 (t, J=7.1 Hz, 1H), 7.04 (d, J=3.6 Hz, 1H), 7.37-7.49 (m, 2H), 7.56 (d, J=3.7 Hz, 1H), 7.67-7.83 (m, 2H), 9.12 (d, J=1.1 Hz, 1H), 9.53 (d, J=7.9 Hz, 1H).

(S—-N-(3-methyl-1-oxo-1-(thiazol-2-ylamino)butan-2-yl)-1H-indole-2-carboxamide (8). 1H NMR (400 MHz, Chloroform-d) δ 1.18 (dd, J=6.7, 14.3 Hz, 6H), 2.32 (h, J=6.8 Hz, 1H), 5.33 (t, J=8.7 Hz, 1H), 7.07-7.19 (m, 3H), 7.26 (d, J=6.8 Hz, 1H), 7.31 (ddd, J=1.1, 7.0, 8.2 Hz, 1H), 7.50 (d, J=8.3 Hz, 1H), 7.63-7.73 (m, 2H), 11.38 (s, 1H), 13.51 (s, 1H).

(S)—N-(1-oxo-1-(thiazol-2-ylamino)propan-2-yl)thiophene-2-carboxamide (9). 1H NMR (500 MHz, Chloroform-d) δ 1.44 (d, J=7.3 Hz, 3H), 4.66 (p, J=7.1 Hz, 1H), 7.19 (dd, J=3.7, 5.0 Hz, 1H), 7.23 (d, J=3.5 Hz, 1H), 7.49 (d, J=3.6 Hz, 1H), 7.79 (dd, J=1.1, 5.0 Hz, 1H), 7.94 (dd, J=1.2, 3.8 Hz, 1H), 8.78 (d, J=6.7 Hz, 1H), 12.29 (s, 1H).

(S)—N-(4-methyl-1-oxo-1-(thiazol-2-ylamino)pentan-2-yl)thiophene-2-carboxamide (10). 1H NMR (400 MHz, Chloroform-d) δ 0.98 (dd, J=1.6, 6.4 Hz, 6H), 1.72-1.86 (m, 2H), 1.87-1.99 (m, 1H), 6.44 (d, J=8.1 Hz, 1H), 7.01 (d, J=3.6 Hz, 1H), 7.10 (dd, J=3.7, 5.0 Hz, 1H), 7.53 (dd, J=1.2, 5.0 Hz, 1H), 7.56-7.64 (m, 2H), 10.69 (s, 1H).

(S)—N-(1-oxo-3-phenyl-1-(thiazol-2-ylamino)propan-2-yl)thiophene-2-carboxamide (11). 1H NMR (500 MHz, DMSO-d6) δ 2.92-3.26 (m, 2H), 4.92 (t, J=8.0 Hz, 1H), 7.12-7.23 (m, 2H), 7.22-7.37 (m, 3H), 7.40-7.48 (m, 2H), 7.51 (dd, J=0.9, 3.4 Hz, 1H), 7.76 (dd, J=1.2, 5.0 Hz, 1H), 7.89 (dd, J=1.2, 3.7 Hz, 1H), 8.87 (d, J=7.9 Hz, 1H), 12.51 (s, 1H).

N-((2S,3S)-3-methyl-1-oxo-1-(thiazol-2-ylamino)pentan-2-yl)thiophene-2-carboxamide (12). 1H NMR (500 MHz, DMSO-d6) δ 0.88 (qd, J=2.2, 7.1 Hz, 5H), 1.27 (dt, J=7.9, 14.1 Hz, 1H), 1.57 (dd, J=8.5, 13.6 Hz, 1H), 1.96-2.10 (m, 1H), 4.52-4.61 (m, 1H), 7.13-7.20 (m, 1H), 7.23 (q, J=3.5, 4.5 Hz, 1H), 7.42-7.65 (m, 1H), 7.79 (dd, J=1.8, 5.2 Hz, 1H), 8.00 (dt, J=1.5, 3.4 Hz, 1H), 8.55-8.71 (m, 1H), 12.35 (s, 1H).

(S)—N-(3-methyl-1-oxo-1-(phenylamino)butan-2-yl)thiophene-2-carboxamide (13). 1H NMR (500 MHz, DMSO-d6) δ 0.99 (dt, J=6.0, 12.0 Hz, 6H), 2.19 (h, J=6.4 Hz, 1H), 4.40 (q, J=7.4, 7.9 Hz, 1H), 7.07 (q, J=6.8 Hz, 1H), 7.17 (q, J=4.8 Hz, 1H), 7.32 (q, J=7.0 Hz, 2H), 7.65 (t, J=6.6 Hz, 2H), 7.77 (d, J=5.4 Hz, 1H), 7.98-8.12 (m, 1H), 8.56 (dd, J=5.2, 8.6 Hz, 1H), 10.21 (d, J=5.2 Hz, 1H).

(S)—N-(3-methyl-1-(oxazol-2-ylamino)-1-oxobutan-2-yl)thiophene-2-carboxamide (14). 1H NMR (400 MHz, Chloroform-d) δ 1.07 (dd, J=6.6, 8.8 Hz, 6H), 2.31 (h, J=6.9 Hz, 1H), 6.98-7.06 (m, 1H), 7.09 (s, 1H), 7.41-7.58 (m, 3H), 7.66 (d, J=3.6 Hz, 1H), 11.50 (s, 1H).

(S)—N-(1-((1,3,4-thiadiazol-2-yl)amino)-3-methyl-1-oxobutan-2-yl)thiophene-2-carboxamide (15). 1H NMR (400 MHz, Acetone-d6) δ 1.08 (dd, J=6.7, 14.8 Hz, 6H), 2.36 (h, J=6.9, 13.8 Hz, 1H), 4.74 (t, J=7.9 Hz, 1H), 7.07-7.20 (m, 1H), 7.64-7.79 (m, 1H), 7.88 (dd, J=1.2, 3.8 Hz, 1H), 7.92-8.05 (m, 1H), 9.08 (s, 1H), 11.95 (s, 1H).

(S)—N-(1-(benzo[d]oxazol-2-ylamino)-3-methyl-1-oxobutan-2-yl)thiophene-2-carboxamide (16). 1H NMR (500 MHz, Chloroform-d) δ 1.09 (dd, J=6.7 Hz, 6H), 2.42 (h, J=6.9, 13.6 Hz, 1H), 4.99 (t, 1H), 7.07 (dd, J=3.7, 5.0 Hz, 1H), 7.25-7.30 (m, 2H), 7.39-7.45 (d, 1H), 7.51 (d, J=1.0, 5.0 Hz, 1H), 7.54-7.62 (m, 1H), 7.70-7.83 (m, 2H).

(thiophene-2-carbonyl)-L-valine (17). S-valine (5 mmol) was dissolved in 5 mLs of 4 M aq NaOH to which 10 mmol of aromatic acyl chloride was added. The mixture was stirred at room temperature for 20 minutes at which point the mixture was placed on ice and acidified with 1 M HCl. From this, a precipitate was formed which was collected by filtration and suspended in EtOAc. This organic solution was then washed 3× with 1 M HCl and dried over Na₂SO₄. The desired product was then purified using flash chromatography (25% EtOAc and 2% HOAc in hexanes). Combined fractions were dried to an oil which was resuspended and dried twice with toluene and once with MeOH yielding product 17. 1H NMR (400 MHz, Chloroform-d) δ 1.07 (dd, J=6.9, 10.2 Hz, 6H), 2.39 (h, J=6.9 Hz, 1H), 4.79 (dd, J=4.9, 8.5 Hz, 1H), 6.69 (d, J=8.5 Hz, 1H), 7.11 (dd, J=3.7, 5.0 Hz, 1H), 7.53 (dd, J=1.1, 5.0 Hz, 1H), 7.63 (dd, J=1.1, 3.8 Hz, 1H), 10.23 (s, 1H).

To synthesize compounds 18-21, compound 17 (lmmol) was dissolved in desired alcohol (8 mLs) to which 500 μL of H₂SO₄ was added. This mixture was refluxed overnight and neutralized with aqueous sodium carbonate. The aqueous basic mixture was extracted multiple times with EtOAc and the combined organic layers were dried over Na₂SO₄. The desired product was then purified by silica chromatography (20% EtOAc in hexanes).

Methyl (thiophene-2-carbonyl)-L-valinate (18). 1H NMR (400 MHz, Chloroform-d) δ 0.99 (dd, J=6.9, 10.6 Hz, 6H), 2.26 (h, J=6.9 Hz, 1H), 4.73 (dd, J=5.0, 8.6 Hz, 1H), 6.51 (d, J=8.6 Hz, 1H), 7.08 (dd, J=3.7, 5.0 Hz, 1H), 7.48 (dd, J=1.1, 5.0 Hz, 1H), 7.55 (dd, J=1.2, 3.7 Hz, 1H).

Ethyl (thiophene-2-carbonyl)-L-valinate (19). 1H NMR (400 MHz, Chloroform-d) δ 1.02 (dd, J=6.9, 11.7 Hz, 6H), 1.34 (t, J=7.1 Hz, 3H), 2.30 (h, J=6.9 Hz, 1H), 4.26 (qq, J=3.6, 7.3 Hz, 2H), 4.75 (dd, J=4.8, 8.6 Hz, 1H), 6.53 (d, J=8.7 Hz, 1H), 7.11 (dd, J=3.7, 5.0 Hz, 1H), 7.51 (dd, J=1.2, 5.0 Hz, 1H), 7.58 (dd, J=1.2, 3.7 Hz, 1H).

Isopropyl (thiophene-2-carbonyl)-L-valinate (20). 1H NMR (400 MHz, Chloroform-d) δ 1.02 (dd, J=6.9, 13.3 Hz, 7H), 1.32 (d, J=6.3 Hz, 7H), 2.30 (h, J=6.9 Hz, 1H), 4.72 (dd, J=4.7, 8.5 Hz, 1H), 5.12 (h, J=6.2 Hz, 1H), 6.54 (d, J=8.6 Hz, 1H), 7.11 (dd, J=3.7, 5.0 Hz, 1H), 7.52 (dd, J=1.2, 5.0 Hz, 1H), 7.58 (dd, J=1.2, 3.7 Hz, 1H).

Butyl (thiophene-2-carbonyl)-L-valinate (21). 1H NMR (400 MHz, Chloroform-d) δ 0.93-1.07 (m, 9H), 1.36-1.49 (m, 2H), 1.62-1.75 (m, 2H), 2.29 (h, J=6.9 Hz, 1H), 4.14-4.26 (m, 2H), 4.72-4.81 (m, 1H), 6.56 (d, J=8.6 Hz, 1H), 7.10 (dd, J=3.7, 5.0 Hz, 1H), 7.50 (dd, J=1.2, 5.0 Hz, 1H), 7.57 (dd, J=1.2, 3.7 Hz, 1H).

### Example 2

### Biological Activities

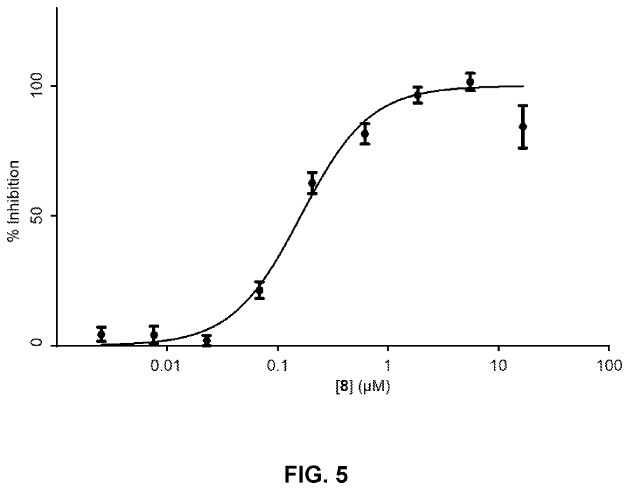

Compound 8 was selected for further investigation because it had the higher potency while maintaining the selectivity of the original hit compound 2. Further experiments were conducted to investigate the potency. Because the values in tables 1-3 were obtained as IC₅₀ values from an assay containing 1 μM protein, the minimum possible IC₅₀ is 500 nM. As the IC₅₀ of 8 for HSPA5 approaches this limit, the titration was repeated with 200 nM HSPA5 SBD (FIG. 5). This indeed showed a decreased IC₅₀ value of 163 nM±17 nM which is greater than the new lower limit of the assay (100 nM). Therefore this IC₅₀ is closer to the actual dissociation constant and more relevant in the analysis of SAR.

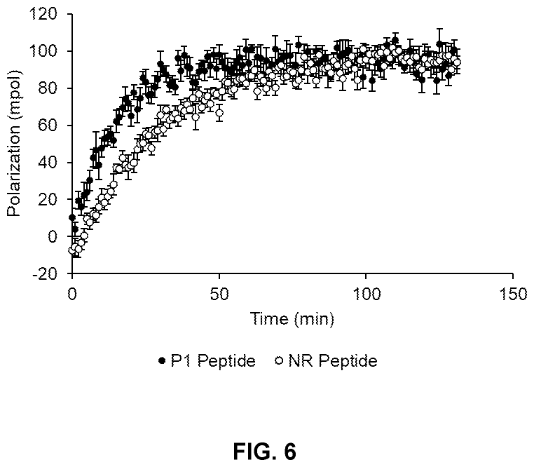

When examining the overall SAR, it became clear that generally few of the molecules inhibited the HSP70-P1 interaction as compared to the HSP70-NR interaction. As the P1 peptide had significantly faster on rates than the NR peptide (FIG. 6) despite them having similar affinity it became important to rule out peptide bias in the activity of the compounds. In general, waiting until the binding between protein, peptide, and inhibitor reaches equilibrium would rule out this kinetic bias. However, having a more direct measurement of compound binding is more powerful than relying on careful measurement times.

To rule out the possibility that the inhibitors were able to compete more efficiently for the NR peptide than the P1 peptide, a fluorescent derivative of 8 (the most potent inhibitor) was synthesized using copper-free click chemistry (compound 22).

Synthesis of Compound 22

Ethyl 5-amino-1H-indole-2-carboxylate. Ethyl 5-nitro-1H-indole-2-carboxylate (5 mmol) was dissolved in 20 mls of 1:1 EtOAc:MeOH. To this mixture was added Pd on carbon (20% by weight) under a hydrogen atmosphere. The reaction was stirred for 36 hours until the staring material was gone by TLC. The Pd catalyst was removed by filtration, solvent was removed, and ethyl 5-amino-1H-indole-2-carboxylate was used without further purification.

Ethyl 5-azido-1H-indole-2-carboxylate. Ethyl 5-amino-1H-indole-2-carboxylate (5 mmol) was dissolved in 6 ml of 16% HCl and cooled in an ice bath. This solution was then treated with NaNO₂ (7.5 mmol) dissolved in 1 ml of water for 30 minutes. This solution was then added to a cooled solution of NaN₃ (10 mmol) and NaOAc (50 mmol) in 15 mL of H₂O. This mixture was then stirred for 30 min on ice and 1 hr at room temperature. The solution was then diluted with water and extracted with CHCl₃ 3×x. The combined organic layers were dried over Na₂SO₄ and the solvent was removed. ethyl 5-azido-1H-indole-2-carboxylate was purified from silica chromatography (25% EtOAc in hexanes).

5-azido-1H-indole-2-carboxylic acid Ethyl 5-azido-1H-indole-2-carboxylate (1 mmol) was dissolved in 4:1 dioxane/H₂O 20 mls to which 5 mL of 4M LiOH was added and the mixture was stirred at room temperature for 12 hours. Once TLC indicated that the reaction was complete, the mixture was acidified with 1 M HCl and the mixture was extracted with EtOAc. The combined organic fractions were dried over Na₂SO₄ and solvent was removed. 5-azido-1H-indole-2-carboxylic acid was used without further purification.

(S)-5-azido-N-(3-methyl-1-oxo-1-(thiazol-2-ylamino)butan-2-yl)-1H-indole carboxamide (compound 22a). 5-azido-1H-indole-2-carboxylic acid (1 mmol) was dissolved in 4 mL of DMF to which 1.5 mmol BOP was added and the mixture was stirred for 30 minutes. To this, 1.1 mmol (S)-2-amino-3-methyl-N-(thiazol-2-yl)butanamide was added along with 3 mmol DIEA. The mixture was stirred overnight at room temperature. The reaction was then diluted with saturated sodium bicarbonate and extracted with EtOAc 3×. The combined organic layers were dried over Na₂SO₄ and the desired product was purified by silica chromatography (50% EtOAc in hexanes).

Compound 22 was prepared by mixing 200 μL of a 10 mM solution of (S)-5-azido-N-(3-methyl-1-oxo-1-(thiazol-2-ylamino)butan-2-yl)-1H-indole-2-carboxamide (22a)in DMSO with 200 μL of a 10 mM solution of carboxyrhodamine-peg4-DBCO. The reaction proceeded for 12 hr at 37° C. and was used as a 5 mM solution without further purification.

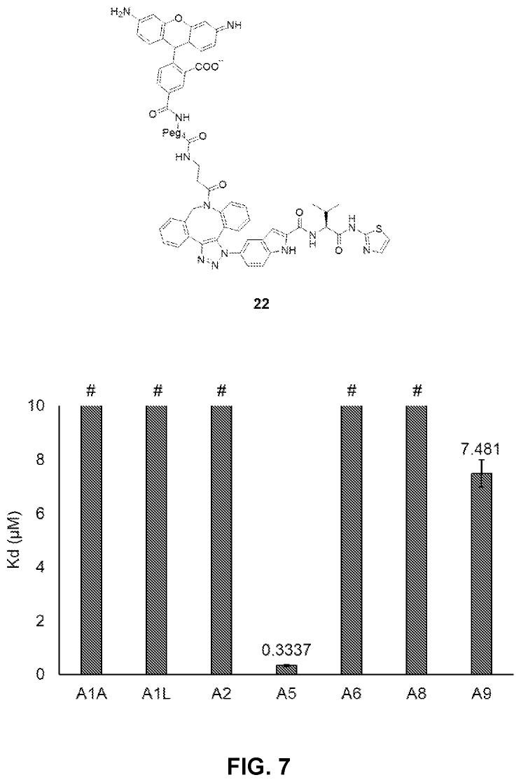

FP was used to measure the affinity of 22 for each HSP70 SBD. As expected, 22 has similar affinity to 8 for each of the HSP70 isoforms and 22 shows no affinity for HSPA1L, which we were unable to measure using P1, NR, or HLA peptides (FIG. 7). Taken together, these data support 8 as a potent and selective binder of HSPA5 as compared to other canonical human HSP70s.

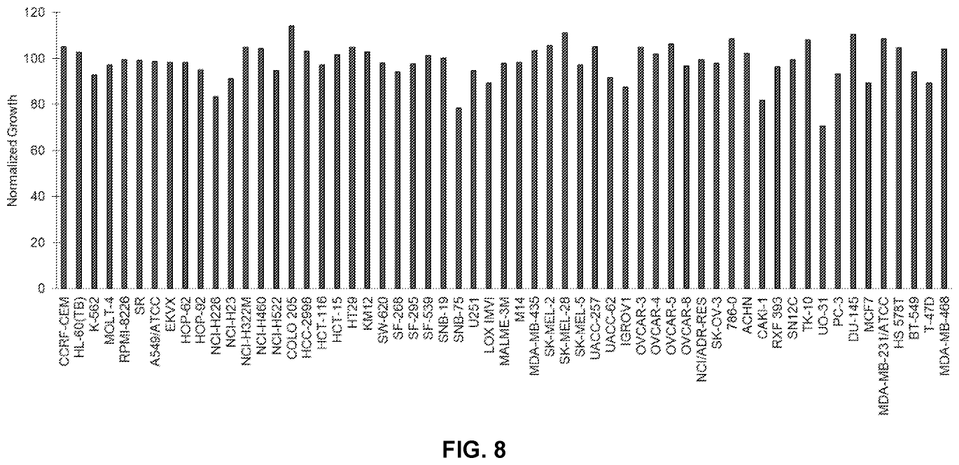

Based on the biochemical data, compound 8 was submitted to the NCI-60 human tumor cell line screen. This platform reports on the toxicity of a given compound in 60 cell lines representing leukemia, melanoma and cancers of the lung, colon, brain, ovary, breast, prostate, and kidney. Compound 8 was tested for growth inhibition against these cell lines at a fixed concentration of 10 μM. These tests did not indicate significant decrease in growth rates in the presence of 8 (FIG. 8). To understand the difference between the biochemical data and the cell line screen, in vitro pharmacokinetic measurements were made of compounds 2, 7, and 8 (Table 4). All three of these compounds have desirable passive membrane permeability and CACO-2 permeability indicating that they can enter cells efficiently. Additionally, none of the compounds show significant binding of human serum albumin, and the primary lead 8 was very stable among human microsomes, being only 12% degraded over 4 hours at 37° C.

TABLE 4

| | | | | Microsome | Caco-2 | Caco-2 |
|---|---|---|---|---|---|---|
| | | | | Stability | Permeability | Permeability |
| | | PAMPA | Serum Binding | (% remaining | B → A | A → B |
| # | Structure | (nm/sec) | (50 μM) | after 4 hours) | (nm/sec) | (nm/sec) |
| 2 | 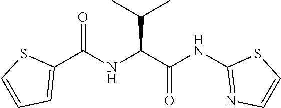 [SMILES: CC(C)[C@H](NC(=O)c1cccs1)C(=O)Nc1nccs1] | 99.8 +/− 20.0 | <10% | N/A | N/A | N/A |
| 7 | 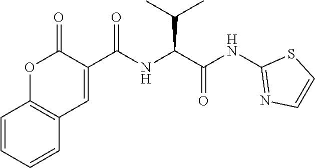 [SMILES: CC(C)[C@H](NC(=O)c1cc2ccccc2oc1=O)C(=O)Nc1nccs1] | 150.3 +/− 10.6 | <10% | N/A | 331.4 +/− 13.2 | 523.1 +/− 12.0 |
| 8 | 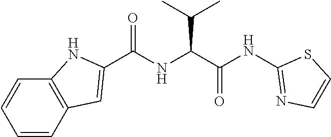 [SMILES: CC(C)[C@H](NC(=O)c1cc2ccccc2[nH]1)C(=O)Nc1nccs1] | 70.9 +/− 21.5 | <10% | 82 +/− 4.6 | 149.5 +/− 46.3 | 175.5 +/− 24.5 |

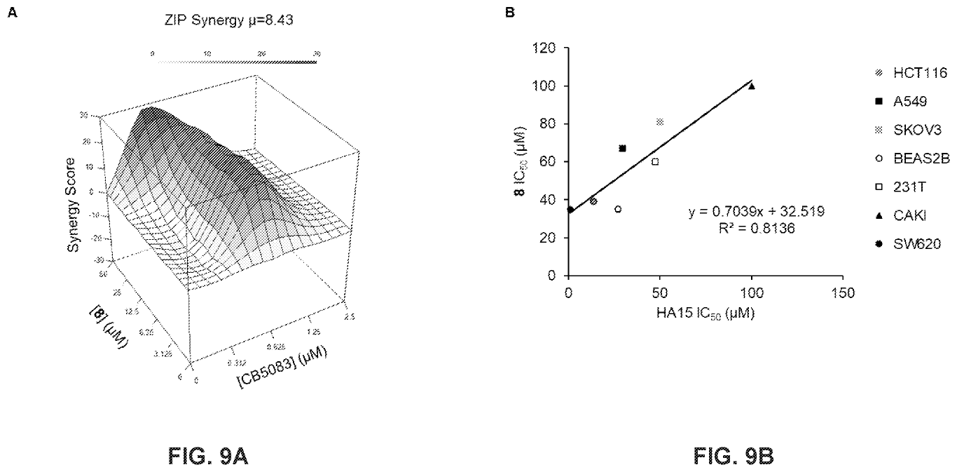

Because HSPA5 functions by maintaining proteostasis in the ER, it can be argued that the lack of growth inhibition could be a result of having too little proteotoxic stress in the ER before HSPA5 inhibition. To investigate this hypothesis, CB5083, a potent p97 inhibitor and inducer of proteotoxic stress in the ER, was added in conjunction with 8 and zero interaction potency (ZIP) synergy was measured. ZIP synergy captures the drug interaction relationships by comparing the change in the potency of the dose—response curves between individual treatments and their combinations without the inherent bias of other synergy measurement such as highest single agent, bliss independence, and Loewe additivity models. When CB5083 and 8 were added to cells together, growth inhibition was observed when both compounds are present a concentration where neither showed activity independently (FIG. 9A). This observation indicates that 8 is active at a concentration near its biochemical Kᵢ, even though it does not inhibit cancer cell growth at these low concentrations.

To further investigate the target of 8, a panel of 7 cell lines were challenged with either 8 or a known HSPA5 inhibitor, HA15. HA15 functions as a selective ATPase inhibitor of HA15 that is toxic to cancer cells without the need for an adjuvant. In these 7 cell lines there is a strong correlation between the IC₅₀ of HA15 and of 8 (FIG. 9B). The Pearson's R² of 0.81 that exists between these compounds indicates that they are likely to share the same target. It is also clear that HA15 is much more potent in cells than 8, suggesting that inhibition of substrate biding to HSPA5 is somehow less stressful to cells than ATPase inhibition of an HSPA5.

In summary, a novel platform for parallel screening of human HSP70 isoforms has revealed a potent and selective class of peptidic inhibitors of substrate binding. Through high throughput screening and lead optimization, 8 has been revealed as a 160 nM inhibitor of HSPA5 and displays greater than 20-fold selectivity over the next most inhibited HSP70 isoform. Although devoid of potent growth inhibition activity, 8 does synergize with the p97 inhibitor CB5083 and correlated with the known HSPA5 inhibitor HA15 in a selected panel of cell lines.

### Example 3

### Additional Compounds and Studies

Materials and Methods

IRE1 dimerization assay. HspA5's induction of IRE1 dimerization was conducted as previously described (1). Briefly, the luminal domain of IRE1 containing the R324C mutation was purified from E. coli and labeled with Oregon Green iodoacetic acid (referred to as IRE1-OG). Separately, the luminal domain of IRE1 containing the S112C mutation was purified from E. coli with the J domain of ERDJ4 fused to the N-terminus and labeled with tamara-maleimide (referred to as J-IRE1-TMR). 1 μM IRE1-OG was incubated with 4 μM J-IRE1-TMR in the dark for 2 hours at room temperature. At this point it was confirmed that the fluorescence of IRE1-OG was quenched. In wells of a black low volume 384 well plate 9 μL of an 8.8 μM HspA5 solution was added. To this, 1 μL of inhibitors or DMSO (vehicle) were added and allowed to bind for 10 minutes at room temperature. Next, 5 μL of the dimerized IRE1 FRET pair was added followed immediately by 5 μL of 40 mM ATP. OG fluorescence (ex: 488 em: 520) was monitored for 15 minutes at room temperature. Fluorescence rate was calculated using linear regression and plotted as a function of concentration of inhibitor.

NMR. A human HspA5 substrate binding domain construct (residues 418-635; HspA5SBD) was subcloned into a pSpeed-His-Smt3 vector to create a His6-Smt3-HspA5SBD construct. The plasmid was transformed into BL21-Codon+ cells and uniformly [15N]-labeled protein was prepared as follows: 1 L cultures were grown in LB at 37° C. to an OD600 of 0.7 and pelleted by a 10 min centrifugation at 5488×g. Cells were then washed and pelleted using an M9 salt solution, excluding all nitrogen and carbon sources. The cell pellet was resuspended in isotopically labeled 15NH4Cl (Cambridge Isotope Laboratories) (1 g/L) M9 minimal media, then incubated with shaking at 37° C. for one hour to allow the recovery of growth and clearance of unlabeled metabolites. Cultures were transferred to 24° C. and protein expression was induced by addition of arabinose to a concentration of 2 g/L. After a 14 h induction period the cells were harvested by centrifugation.

Cell pellets were resuspended in ice-cold lysis buffer (25 mM Tris-HCl pH 8.0, 150 mM NaCl) and lysed by high-pressure cell homogenization (Microfluidics LM10 Microfluidizer). The bacterial lysate was clarified by centrifugation at 15120×g for 40 min at 4° C. The supernatant was loaded onto a high density cobalt resin (GoldBio) column equilibrated with lysis buffer. The His6-Smt3-tagged protein was eluted with lysis buffer and a gradient of increasing imidazole concentration. Fractions containing the target protein were pooled and dialyzed against lysis buffer overnight to remove imidazole. The protein was then removed from dialysis and unfolded in 8M urea to remove clients bound to the HSPA5SBD. The unfolded protein was incubated with high density cobalt resin (GoldBio) equilibrated with Urea+lysis buffer. The His6-Smt3-tagged protein was eluted with Urea+lysis buffer and a gradient of increasing imidazole concentration. Fractions containing clean protein were pooled and subject to multiple rounds of 4 hour dialyses into lysis buffer to remove urea. Protein was then collected and the His6-Smt3 tag was cut by His6-Ulp1 protease catalytic domain (403-621) (1:500 Ulp:Protein [w/w]) in cutting buffer (25 mM Tris-HCl pH 8.0, 150 mM NaCl, 0.1% Tween-20, 2 mM DTT) overnight at 4° C. Protein was collected, and His6-Ulp1 was recaptured through incubation with Ni-NTA Agarose resin (Qiagen) equilibrated with cutting buffer. Protein was then concentrated with Pierce Protein Concentrators (Thermo Scientific), and an equal volume of urea was added. This was further purified by size exclusion chromatography (HiLoad 26/600, Superdex 75 pg) equilibrated in Urea+NMR buffer (20 mM Hepes, pH 6.8). Fractions containing pure protein were collected and subject to multiple rounds of 4 hour dialyses in NMR buffer to remove urea. Protein was removed from dialysis and concentrated with Pierce Protein Concentrators (Thermo Scientific), and further buffer exchanged into NMR buffer using a Zeba Spin Desalting column. Purified HspA5SBD was concentrated to 150 μM. For long-term storage, the protein was flash frozen in liquid nitrogen and stored at −80° C.

NMR Collection and Processing. 2D [1H, 15N] TROSY spectra were recorded on 150 μM [15N]-HspA5SBD with and without addition of 180 μM 25 (each containing 2.5% d6-DMSO). 15N-HSQCs were collected at 303 K on a Bruker Avance 800 MHz spectrometer equipped with a TCI HCN z-gradient cryoprobe. All NMR spectra were processed with Topspin 4.0.6 (Bruker).

Cellular Synergy. U251 glioblastoma cells were cultured with the indicated treatment or combination of treatments and their confluence was measured every 24 hours for 72 hours using an incucyte imager. The growth rate was then calculated and plotted as a function of time. Growth rates were then calculate as the slope of the linear fit.

Synthesis. The following compounds were synthesized from commercial material according to scheme 1.

(S)—N-(1-((4,5-dimethylthiazol-2-yl)amino)-3-methyl-1-oxobutan-2-yl)thiophene-2-carboxamide (23): 1H NMR (400 MHz, Acetone) δ 1.07 (dd, J=6.8, 5.6 Hz, 6H), 2.17 (s, 3H), 2.28 (s, 3H) 2.38-2.29 (m, 1H), 4.76-4.67 (m, 1H), 7.15 (dd, J=5.0, 3.8 Hz, 1H), 7.71 (dd, J=5.0, 1.1 Hz, 1H), 7.84 (d, J=8.5 Hz, 1H), 7.89 (dd, J=3.8, 1.1 Hz, 1H), 10.96 (s, 1H).

(S)-5-acetyl-N-(3-methyl-1-oxo-1-(thiazol-2-ylamino)butan-2-yl)-1H-indole-2-carboxamide (24): 1H NMR (500 MHz, CDCl3) δ 1.20-1.04 (dd, J=6.8, 5.6 Hz, 6H), 2.47-2.32 (m, 1H), 2.96 (s, 3H), 5.32 (t, J=9.2 Hz, 1H), 7.30 (d, J=3.5 Hz, 1H), 7.48-7.42 (m, 1H), 7.50 (m, J=2.2, 0.9 Hz, 1H), 7.60-7.55 (m, 1H), 7.85-7.77 (m, 1H), 8.08-7.98 (m, 1H), 8.13 (m, J=13.6, 2.0 Hz, 1H), 8.49 (d, J=9.3 9.22 (s, 1H), Hz, 1H), 11.96 (s, 1H).

(S)—N-(1-(benzo[d]thiazol-2-ylamino)-3-methyl-1-oxobutan-2-yl)thiophene-2-carboxamide (25): 1H NMR (400 MHz, Acetone) δ 1.12 (dd, J=6.8, 5.6 Hz, 6H) 2.43 (dq, J=13.9, 6.9 Hz, 1H), 4.76 (d, J=7.6 Hz, 1H), 7.17 (dd, J=5.0, 3.8 Hz, 1H), 7.36 (m, 1H), 7.47 (m, 1H), 7.80-7.70 (m, 2H), 8.05-7.88 (m, 2H).

Activities of compounds 8, 23, 24, and 25 against HspA5 are listed in Table 5.

TABLE 5

| | | A5 |
|---|---|---|
| | | IC₅₀ |
| # | Structure | (μM) |
| 8 | 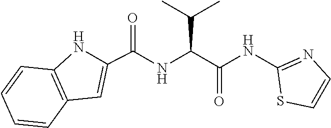 [SMILES: CC(C)[C@H](NC(=O)c1cc2ccccc2[nH]1)C(=O)Nc1nccs1] | 0.59 ± 0.06 |
| 23 | 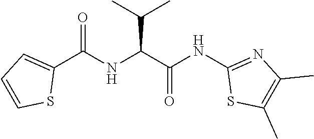 [SMILES: Cc1nc(NC(=O)[C@@H](NC(=O)c2cccs2)C(C)C)sc1C] | 1.4 ± 0.3 |
| 24 | 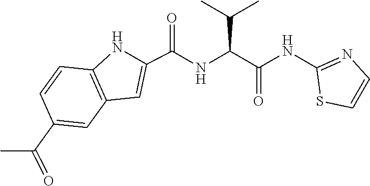 [SMILES: CC(=O)c1ccc2[nH]c(C(=O)N[C@H](C(=O)Nc3nccs3)C(C)C)cc2c1] | 0.43 ± 0.09 |
| 25 | 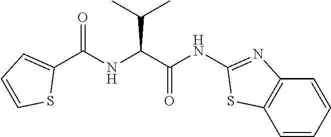 [SMILES: CC(C)[C@H](NC(=O)c1cccs1)C(=O)Nc1nc2ccccc2s1] | 20.3 ± 3 |

In addition to compound 8, compounds 23, 24, and 25 provide further insight towards their binding site in HspA5. Remarkably, compound 24 is much more soluble than compound 8, allowing for more complex experiments to be conducted.

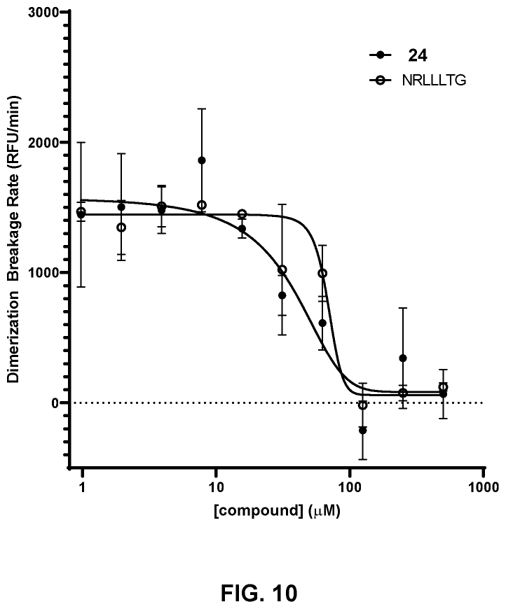

A reported assay allows for the study of HspA5 in a more cellularly relevant context by measuring its ability to block IRE1 dimerization in the presence of a cofactor (Amin-Wetzel N et al., Elife, 2019 Dec. 24; 8). Unfortunately, this assay requires a large amount of protein to generate a significant signal. Because of this, the IC₅₀ of 24 is higher than in the peptide binding assay, but 24 demonstrated equal capacity in blocking HspA5 as compared to the NRLLLTG peptide (FIG. 10). This indicates that the apparent lack of activity is due to the assay conditions and not to a poor compound. Given that the Kd of the NRLLTG peptide is known to be about 100 nM, it is reasonable to conclude that both compound 24 and the peptide are acting at the minimum concentration possible in the assay, which illustrates that 24 is indeed active against the functional HspA5 machinery.

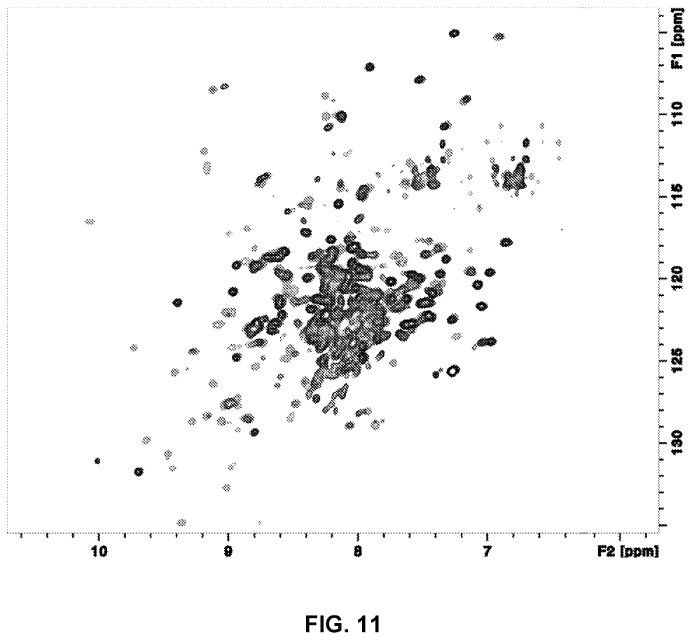

In order to validate binding of compound 24 to the substrate biding domain (SBD) of HSPA5, we produced ¹⁵N-labeled HSPA5 SBD protein and performed heteronuclear single quantum coherence (HSQC) experiments of the protein with and without 24 (FIG. 11). The data show that the SBD is relatively unstable in the absence of a ligand. Upon addition of 24, the formation of several distinct peaks was observed, indicating that 24 binds to HSPA5 in a manner that stabilizes its structure.

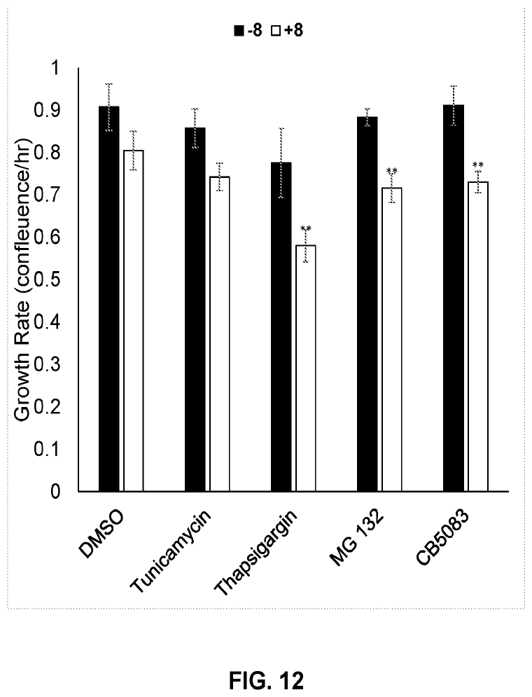

To further expand our understanding of the cellular activity of 8, the compound was tested in an U251 glioblastoma model due to its high expression of HSPA5 and relative sensitivity to the compound. Synergy between 8 and various compounds that induce proteotoxic stress in the endoplasmic reticulum was examined. In this model, it was observed that 8 inhibits growth of these cells in a synergistic manner with MG132, thapsigargin, and CB5082 (FIG. 12).

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A compound of formula (I′), or a pharmaceutically acceptable salt thereof,

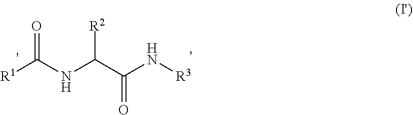

wherein

R¹′ is an aryl or a heteroaryl, wherein R¹′ is optionally substituted with one or more R^{a}ζ;

R²′ is C₁₋₆ alkyl, aryl, heteroaryl, C₁₋₄ alkylene-aryl, or C₁₋₄ alkylene-heteroaryl, wherein R²′ is optionally substituted with one or more R^{b}′;

R³′ is an aryl or a heteroaryl, wherein R³′ is optionally substituted with one or more R^{c}ζ;

R^{a}′, R^{b}′, and R^{c}′ at each occurrence are independently halogen, —CN, nitro, N₃, —SO₂NH₂, or —Y—R^{Y};

Y is bond, O, NH, C(O), OC(O), C(O)NH, or S; and

R^{Y} is H, alkyl, alkenyl, alkynyl, alkoxy, cycloalkyl, aryl, heteroaryl, or heterocyclyl, wherein R^{Y} is optionally substituted;

provided that the compound is not N-(3-methyl-1-oxo-1-(thiazol-2-ylamino)butan-2-yl)thiophene-2-carboxamide.

Clause 2. The compound of clause 1, wherein the compound is compound of formula (I′-a), or a pharmaceutically acceptable salt thereof,

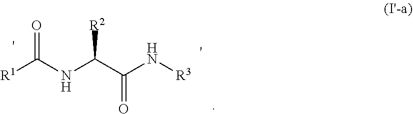

Clause3. The compound of any one of clauses 1-2, wherein R¹ζ is

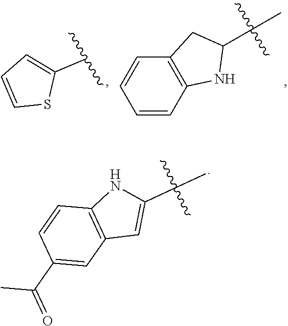

Clause 4. The compound of any one of clauses 1-3, wherein R²′ is C₂₋₄alkyl or benzyl.

Clause 5. The compound of any one of clauses 1-4, wherein R³′ is

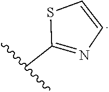 [SMILES: *c1nccs1]

Clause 6. The compound of clause 1, wherein the compound is selected from the group consisting of

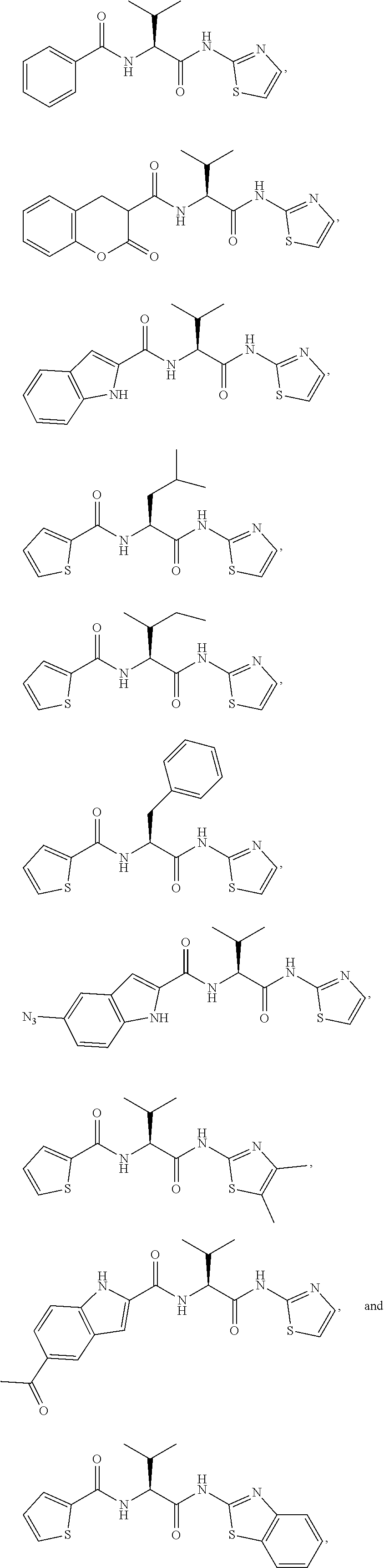

or a pharmaceutically acceptable salt thereof.

Clause 7. The compound of any one of clauses 1-6, wherein the compound is selected from the group consisting of

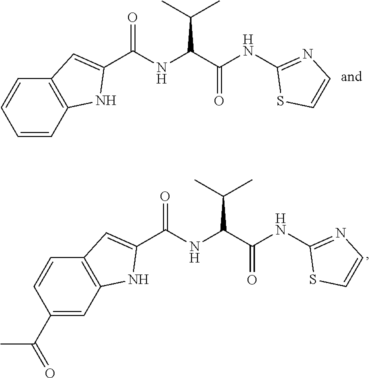

or a pharmaceutically acceptable salt thereof.

Clause 8. A compound, which is an enantiomerically enriched

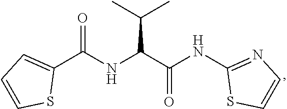

or a pharmaceutically acceptable salt thereof.

Clause 9. The compound of clause 8, wherein the compound has an enantiomeric excess (ee) value of at least 90%.

Clause 10. A pharmaceutical composition comprising a compound of clause 1 or 8, or a pharmaceutically acceptable salt thereof, and a pharmaceutically acceptable carrier.

Clause 11. A method for inhibiting a heat shock protein 70 (HSP70) comprising contacting the HSP70 with a pharmaceutically effective amount of a compound of formula (I), or a pharmaceutically acceptable salt thereof,

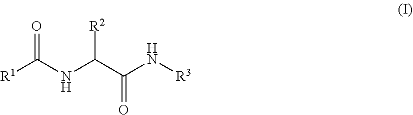

wherein

R¹ is an aryl or a heteroaryl, wherein R¹ is optionally substituted with one or more R^{a};

R² is C₁₋₆alkyl, aryl, heteroaryl, C₁₋₄alkylene-aryl, or C₁₋₄alkylene-heteroaryl, wherein R² is optionally substituted with one or more R^{b};

R³ is an aryl or a heteroaryl, wherein R³ is optionally substituted with one or more R^{c};

R^{a}, R^{b}, and R^{c} at each occurrence are independently halogen, —CN, nitro, N₃, —SO₂NH₂, or —X—R^{X},

X is bond, O, NH, C(O), OC(O), C(O)NH, or S; and

R^{X} is H, alkyl, alkenyl, alkynyl, alkoxy, cycloalkyl, aryl, heteroaryl, or heterocyclyl, wherein R^{X} is optionally substituted.

Clause12. The method of clause 11, wherein the compound is compound of formula (I-a), or a pharmaceutically acceptable salt thereof,

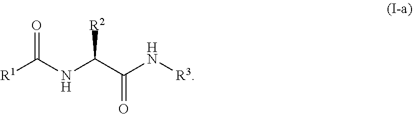

Clause 13. The method of any one of clauses 11-12, where R¹ is

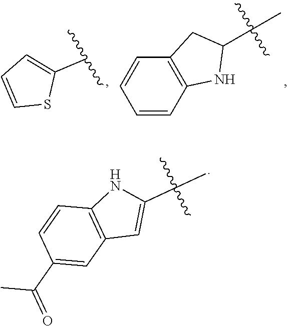

Clause 14. The method of any one of clauses 11-13, wherein R² is C₂₋₄alkyl or benzyl.

Clause 15. The method of any one of clauses 11-14, wherein R³ is

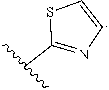 [SMILES: *c1nccs1]

Clause 16. The method of clause 11, wherein the compound is selected from the group consisting of

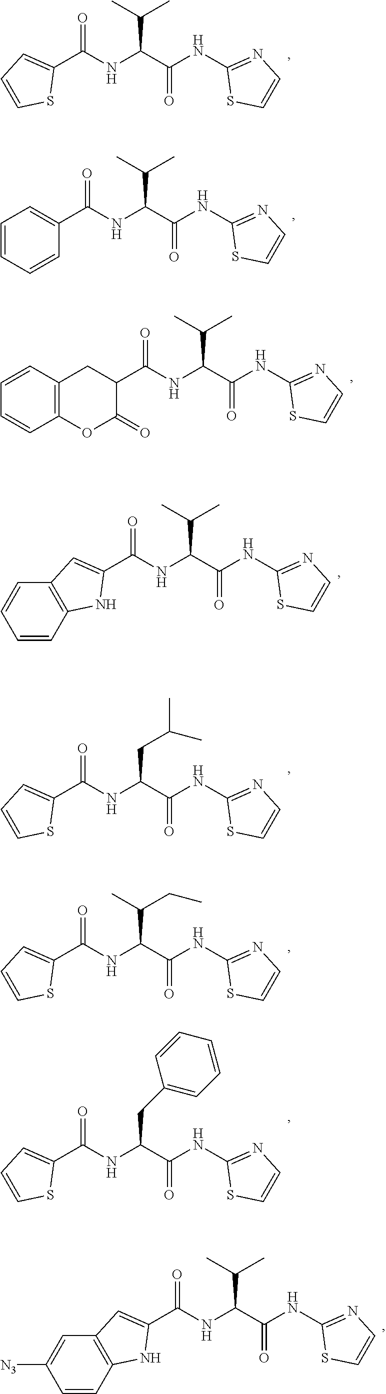

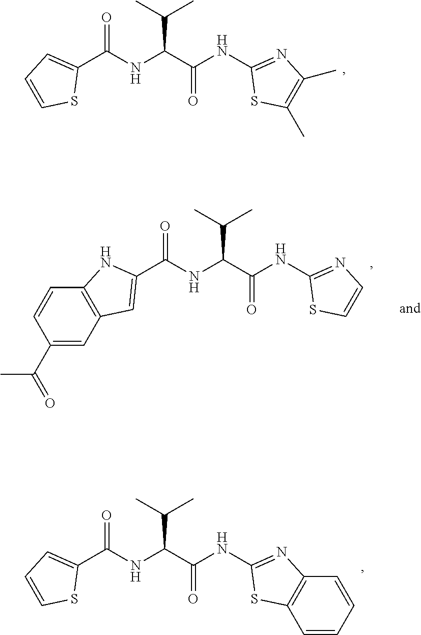

or a pharmaceutically acceptable salt thereof.

Clause 17. The method of any one of clauses 11-16, wherein the compound is selected from the group consisting of

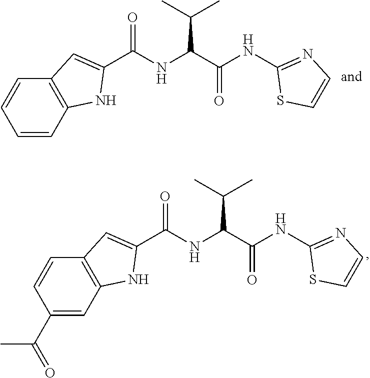

or a pharmaceutically acceptable salt thereof.

Clause 18. The method of any one of clauses 11-17, wherein the HSP70 comprises HSPA5.

Clause 19. The method of any one of clauses 11-18, further comprising contacting the HSP70 with an additional active agent.

Clause 20. The method of clause 19, wherein the additional active agent is CB5083.

Clause 21. A method for treating a disease characterized by overexpression of a heat shock protein 70 (HSP70) comprising administrating to a subject in need thereof a pharmaceutically effective amount of a compound of formula (I), or a pharmaceutically acceptable salt thereof,

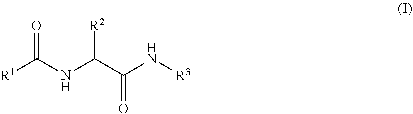

wherein

R¹ is an aryl or a heteroaryl, wherein R¹ is optionally substituted with one or more R^{a};

R² is C₁₋₆alkyl, aryl, heteroaryl, C₁₋₄alkylene-aryl, or C₁₋₄alkylene-heteroaryl, wherein R² is optionally substituted with one or more R^{b};

R³ is an aryl or a heteroaryl, wherein R³ is optionally substituted with one or more R^{c};

R^{a}, R^{b}, and R^{c} at each occurrence are independently halogen, —CN, nitro, N₃, —SO₂NH₂, or —X—R^{X},

X is bond, O, NH, C(O), OC(O), C(O)NH, or S; and

R^{X} is H, alkyl, alkenyl, alkynyl, alkoxy, cycloalkyl, aryl, heteroaryl, or heterocyclyl, wherein R^{X} is optionally substituted.

Clause 22. The method of clause 21, wherein the compound is compound of formula (I-a), or a pharmaceutically acceptable salt thereof,

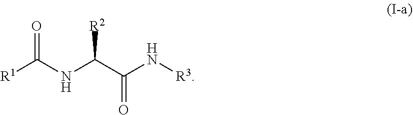

Clause 23. The method of any one of clauses 21-22, wherein R¹ is

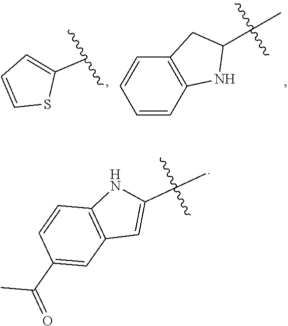

Clause 24. The method of any one of clauses 21-23, wherein R² is C₂₋₄alkyl or benzyl.

Clause 25. The method of any one of clauses 21-24, wherein R³ is

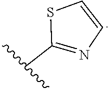 [SMILES: *c1nccs1]

Clause 26. The method of clause 21, wherein the compound is selected from the group consisting of

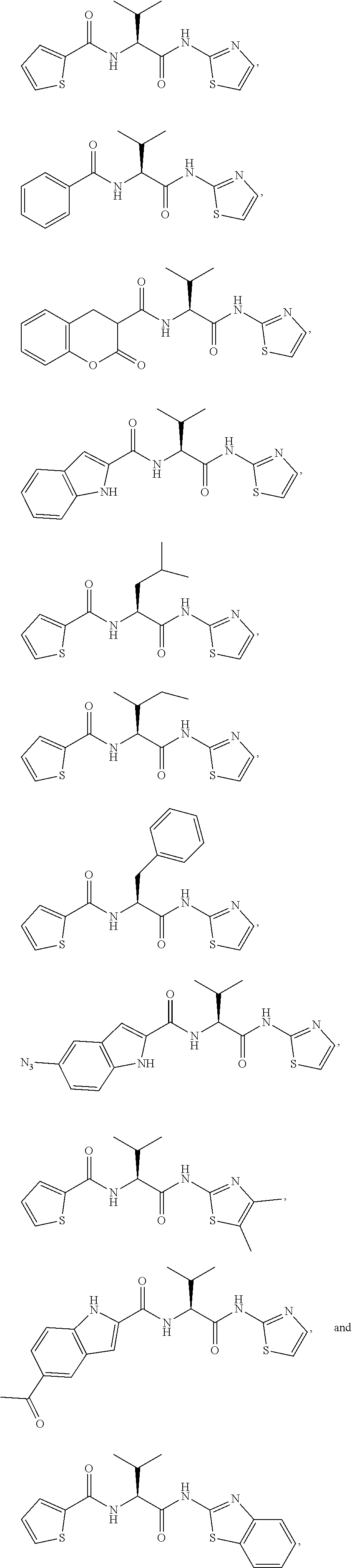

or a pharmaceutically acceptable salt thereof.

Clause 27. The method of any one of clauses 21-26, wherein the compound is selected from the group consisting of

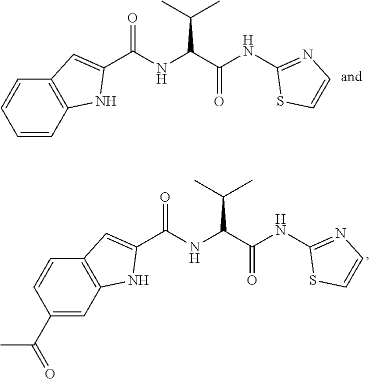

or a pharmaceutically acceptable salt thereof.

Clause 28. The method of any one of clauses 21-27, wherein the HSP70 comprises HSPA5.

Clause 29. The method of any one of clauses 21-28, wherein the disease is cancer.

Clause 30. The method of clause 29, wherein the cancer is bladder cancer, breast cancer, cervical cancer, colorectal cancer, esophageal cancer, glioblastoma, endometrial cancer, leukemia, liver cancer, lung cancer, mantle cell lymphoma, melanoma, multiple myeloma, oral cancer, ovarian cancer, prostate cancer, pancreatic cancer, renal cancer, stomach cancer, testicular cancer, thyroid cancer, or a combination thereof.

Clause 31. The method of clause 30, wherein the cancer is breast cancer, colorectal cancer, lung cancer, renal cancer, or a combination thereof

Clause 32. The method of any one of clauses 21-31, further comprising administrating to the subject an additional active agent.

Clause 33. The method of clause 32, wherein the additional active agent is CB5083.

### REFERENCES

(1) Rosenzweig, R.; Nillegoda, N. B.; Mayer, M. P.; Bukau, B. The Hsp70 Chaperone Network. Nature Reviews Molecular Cell Biology. Nature Publishing Group Nov. 1, 2019, pp 665-680. https://doi.org/10.1038/s41580-019-0133-3.
(2) Daugaard, M.; Rohde, M.; Jäättelä, M. The Heat Shock Protein 70 Family: Highly Homologous Proteins with Overlapping and Distinct Functions. FEBS Lett. 2007, 581 (19), 3702-3710. https://doi.org/10.1016/j.febslet.2007.05.039.
(3) Murphy, M. E. The HSP70 Family and Cancer. Carcinogenesis 2013, 34 (6), 1181-1188. https://doi.org/10.1093/carcin/bgt111.
(4) Yang, J.; Nune, M.; Zong, Y.; Zhou, L.; Liu, Q. Close and Allosteric Opening of the Polypeptide-Binding Site in a Human Hsp70 Chaperone BiP. Structure 2015, 23 (12), 2191-2203. https://doi.org/10.1016/j.str.2015.10.012.
(5) Flaherty, K. M.; DeLuca-Flaherty, C.; McKay, D. B. Three-Dimensional Structure of the ATPase Fragment of a 70K Heat-Shock Cognate Protein. Nature 1990, 346, 623-628.
(6) Chiappori, F.; Merelli, I.; Milanesi, L.; Colombo, G.; Morra, G. An Atomistic View of Hsp70 Allosteric Crosstalk: From the Nucleotide to the Substrate Binding Domain and Back. Sci. Rep. 2016, 6 (1), 23474. https://doi.org/10.1038/srep23474.
(7) Kityk, R.; Kopp, J.; Mayer, M. P. Molecular Mechanism of J-Domain-Triggered ATP Hydrolysis by Hsp70 Chaperones. Mol. Cell 2018, 69 (2), 227-237.e4. https://doi.org/10.1016/J.MOLCEL.2017.12.003.
(8) Nillegoda, N. B.; Wentink, A. S.; Bukau, B. Protein Disaggregation in Multicellular Organisms. Trends in Biochemical Sciences. Elsevier Ltd Apr. 1, 2018, pp 285-300. https://doi.org/10.1016/j.tibs.2018.02.003.
(9) Craig, E. A. Hsp70 at the Membrane: Driving Protein Translocation. BMC Biology. BioMed Central Ltd. Jan. 17, 2018, p 11. https://doi.org/10.1186/s12915-0017-0474-3.
(10) Fernandez-Fernandez, M. R.; Gragera, M.; Ochoa-Ibarrola, L.; Quintana-Gallardo, L.; Valpuesta, J. M. Hsp70-a Master Regulator in Protein Degradation. FEBS Letters. Wiley Blackwell Sep. 1, 2017, pp 2648-2660. https://doi.org/10.1002/1873-3468.12751.
(11) Rohde, M.; Daugaard, M.; Jensen, M. H.; Helin, K.; Nylandsted, J.; Jäättelä, M. Members of the Heat-Shock Protein 70 Family Promote Cancer Cell Growth by Distinct Mechanisms. Genes Dev. 2005, 19 (5), 570-582. https://doi.org/10.1101/gad.305405.
(12) Taldone, T.; Patel, H. J.; Bolaender, A.; Patel, M. R.; Chiosis, G. Protein Chaperones: A Composition of Matter Review (2008-2013). Expert Opin. Ther. Pat. 2014, 24 (5), 501-518. https://doi.org/10.1517/13543776.2014.887681.
(13) Balchin, D.; Hayer-Hartl, M.; Hartl, F. U. In Vivo Aspects of Protein Folding and Quality Control. Science. American Association for the Advancement of Science Jul. 1, 2016, p aac4354. https://doi.org/10.1126/science.aac4354.
(14) Martin, M. D.; Baker, J. D.; Suntharalingam, A.; Nordhues, B. A.; Shelton, L. B.; Zheng, D.; Sabbagh, J. J.; Haystead, T. A. J.; Gestwicki, J. E.; Dickey, C. A. Inhibition of Both Hsp70 Activity and Tau Aggregation in Vitro Best Predicts Tau Lowering Activity of Small Molecules. ACS Chem. Biol. 2016, 11 (7), 2041-2048. https://doi.org/10.102¹/acschembio.6b00223.
(15) The Human Protein Atlas https://www.proteinatlas.org/ (accessed Feb.18, 2020).
(16) Aguilar-Rodriguez, J.; Sabater-Muñoz, B.; Montagud-Martinez, R.; Berlanga, V.; Alvarez-Ponce, D.; Wagner, A.; Fares, M. A. The Molecular Chaperone DnaK Is a Source of Mutational Robustness. Genome Biol. Evol. 2016, 8 (9), 2979-2991. https://doi.org/10.1093/gbe/evw176.
(17) Cesa, L. C.; Shao, H.; Srinivasan, S. R.; Tse, E.; Jain, C.; Zuiderweg, E. R. P.; Southworth, D. R.; Mapp, A. K.; Gestwicki, J. E. X-Linked Inhibitor of Apoptosis Protein (XIAP) Is a Client of Heat Shock Protein 70 (Hsp70) and a Biomarker of Its Inhibition. Journal of Biological Chemistry. American Society for Biochemistry and Molecular Biology Inc. 2018, pp 2370-2380. https://doi.org/10.1074/jbc.RA117.000634.
(18) Boysen, M.; Kityk, R.; Mayer, M. P. Hsp70- and Hsp90-Mediated Regulation of the Conformation of P53 DNA Binding Domain and P53 Cancer Variants. Mol. Cell 2019, 74 (4), 831-843.e4. https://doi.org/10.1016/j.molce1.2019.03.032.
(19) Hupp, T. R.; Meek, D. W.; Midgley, C. A.; Lane, D. P. Regulation of the Specific DNA Binding Function of P53. Cell 1992, 71 (5), 875-886. https://doi.org/10.1016/0092-8674(92)90562-Q.
(20) Hansen, S.; Hupp, T. R.; Lane, D. P. Allosteric Regulation of the Thermostability and DNA Binding Activity of Human P53 by Specific Interacting Proteins. J. Biol. Chem. 1996, 271 (7), 3917-3924. https://doi.org/10.1074/jbc.271.7.3917.
(21) Tanaka, M.; Mun, S.; Harada, A.; Ohkawa, Y.; Inagaki, A.; Sano, S.; Takahashi, K.; Izumi, Y.; Osada-Oka, M.; Wanibuchi, H.; et al. Hsc70 Contributes to Cancer Cell Survival by Preventing Rab1A Degradation under Stress Conditions. PLoS One 2014, 9 (5). https://doi.org/10.1371/journal.pone.0096785.
(22) Ambrose, A. J.; Santos, E. A.; Jimenez, P. C.; Rocha, D. D.; Wilke, D. V.; Beuzer, P.; Axelrod, J.; Kumar Kanduluru, A.; Fuchs, P. L.; Cang, H.; et al. Ritterostatin G _{N} 1 _{N}, a Cephalostatin-Ritterazine Bis-Steroidal Pyrazine Hybrid, Selectively Targets GRP78. ChemBioChem 2017, 18 (6), 506-510. https://doi.org/10.1002/cbic.201600669.

(23) Rodina, A.; Patel, P. D.; Kang, Y.; Patel, Y.; Baaklini, I.; Wong, M. J. H.; Taldone, T.; Yan, P.; Yang, C.; Maharaj, R.; et al. Identification of an Allosteric Pocket on Human Hsp70 Reveals a Mode of Inhibition of This Therapeutically Important Protein. Chem. Biol. 2013, 20 (12), 1469-1480. https://doi.org/10.1016/j.chembio1.2013.10.008.
(24) Cerezo, M.; Lehraiki, A.; Millet, A.; Rouaud, F.; Plaisant, M.; Jaune, E.; Botton, T.; Ronco, C.; Abbe, P.; Amdouni, H.; et al. Compounds Triggering ER Stress Exert Anti-Melanoma Effects and Overcome BRAF Inhibitor Resistance. Cancer Cell 2016, 29 (6), 805-819. https://doi.org/10.1016/J.CCELL.2016.04.013.
(25) A. Assimon, V.; T. Gillies, A.; N. Rauch, J.; E. Gestwicki, J. Hsp70 Protein Complexes as Drug Targets. Curr. Pharm. Des. 2012, 19 (3), 404-417. https://doi.org/10.2174/138161213804143699.
(26) Hunt, C. R.; Dix, D. J.; Sharma, G. G.; Pandita, R. K.; Gupta, A.; Funk, M.; Pandita, T. K. Genomic Instability and Enhanced Radiosensitivity in Hsp70.1- and Hsp70.3-Deficient Mice. Mol. Cell. Biol. 2004, 24 (2), 899-911. https://doi.org/10.1128/mcb.24.2.899-911.2004.
(27) Wisniewska, M.; Karlberg, T.; Lehtio, L.; Johansson, I.; Kotenyova, T.; Moche, M.;

Schüller, H. Crystal Structures of the ATPase Domains of Four Human Hsp70 Isoforms: HSPA1L/Hsp70-Hom, HSPA2/Hsp70-2, HSPA6/Hsp70B′, and HSPA5/BiP/GRP78. PLoS One 2010, 5 (1), e8625. https://doi.org/10.1371/journal.pone.0008625.
(28) Zuiderweg, E. R. P.; Hightower, L. E.; Gestwicki, J. E. The Remarkable Multivalency of the Hsp70 Chaperones. Cell Stress Chaperones 2017, 22 (2), 173-189. https://doi.org/10.1007/s12192-017-0776-y.
(29) Fourieso, A. M.; Sambrookl, J. F.; Gethingson, M.-J. H. Common and Divergent Peptide Binding Specificities of Hsp70 Molecular Chaperones*; 1994; Vol. 269.
(30) Takenaka, I. M.; Leung, S. M.; McAndrew, S. J.; Brown, J. P.; Hightower, L. E. Hsc70-Binding Peptides Selected from a Phage Display Peptide Library That Resemble Organellar Targeting Sequences. J. Biol. Chem. 1995, 270 (34), 19839-19844. https://doi.org/10.1074/jbc.270.34.19839.
(31) Taylor, I. R.; Ahmad, A.; Wu, T.; Nordhues, B. A.; Bhullar, A.; Gestwicki, J. E.; Zuiderweg, E. R. P. The Disorderly Conduct of Hsc70 and Its Interaction with the Alzheimer's-Related Tau Protein. J. Biol. Chem. 2018, 293 (27), 10796-10809. https://doi.org/10.1074/jbc.RA118.002234.
(32) Kang, Y.; Taldone, T.; Clement, C. C.; Fewell, S. W.; Aguirre, J.; Brodsky, J. L.; Chiosis, G. Design of a Fluorescence Polarization Assay Platform for the Study of Human Hsp70. Bioorg. Med. Chem. Lett. 2008, 18 (13), 3749-3751. https://doi.org/10.1016/j.bmc1.2008.05.046.
(33) Schlecht, R.; Scholz, S. R.; Dahmen, H.; Wegener, A.; Sirrenberg, C.; Musil, D.; Bomke, J.; Eggenweiler, H.-M.; Mayer, M. P.; Bukau, B. Functional Analysis of Hsp70 Inhibitors. PLoS One 2013, 8 (11), e78443. https://doi.org/10.1371/journal.pone.0078443.
(34) Williamson, D. S.; Borgognoni, J.; Clay, A.; Daniels, Z.; Dokurno, P.; Drysdale, M. J.; Foloppe, N.; Francis, G. L.; Graham, C. J.; Howes, R.; et al. Novel Adenosine-Derived Inhibitors of 70 KDa Heat Shock Protein, Discovered Through Structure-Based Design †. J. Med. Chem. 2009, 52 (6), 1510-1513. https://doi.org/10.1021/jm801627a.
(35) Ambrose, A. J.; Zerio, C. J.; Sivinski, J.; Schmidlin, C. J.; Shi, T.; Ross, A. B.; Widrick, K. J.; Johnson, S. M.; Zhang, D. D.; Chapman, E. A High Throughput Substrate Binding Assay Reveals Hexachlorophene as an Inhibitor of the ER-Resident HSP70 Chaperone GRP78. Bioorg. Med. Chem. Lett. 2019, 29 (14), 1689-1693. https://doi.org/10.1016/j.bmc1.2019.05.041.
(36) Yadav, B.; Wennerberg, K.; Aittokallio, T.; Tang, J. Searching for Drug Synergy in Complex Dose-Response Landscapes Using an Interaction Potency Model. Comput. Struct. Biotechnol. J. 1 2015, 13, 504-513. https://doi.org/10.1016/j.csbj.2015.09.001.
(37) Er, J. C.; Vendrell, M.; Tang, M. K.; Zhai, D.; Chang, Y.-T. Fluorescent Dye Cocktail for Multiplex Drug-Site Mapping on Human Serum Albumin. ACS Comb. Sci. 2013, 15 (9), 452-457. https://doi.org/10.1021/co400060b.
(38) He, L.; Kulesskiy, E.; Saarela, J.; Turunen, L.; Wennerberg, K.; Aittokallio, T.; Tang, J. Methods for High-Throughput Drug Combination Screening and Synergy Scoring. In Methods in Molecular Biology; Humana Press Inc., 2018; Vol. 1711, pp 351-398. https://doi.org/10.1007/978-1-4939-7493-1_17.
(39) Amin-Wetzel N, Neidhardt L, Yan Y, Mayer MP, Ron D. Unstructured regions in

IRE1α specify BiP-mediated destabilisation of the luminal domain dimer and repression of the UPR. Elife. 2019 Dec 24;8.

1. A compound of formula (I′), or a pharmaceutically acceptable salt thereof,
wherein 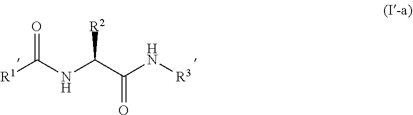
R¹′ is an aryl or a heteroaryl, wherein R¹′ is optionally substituted with one or more R^{a}′;
R²′ is C₁₋₆alkyl, aryl, heteroaryl, C₁₀₋₄alkylene-aryl, or C₁₀₋₄alkylene-heteroaryl, wherein R²′ is optionally substituted with one or more R^{b}′;
R³′ is an aryl or a heteroaryl, wherein R³′ is optionally substituted with one or more R^{c}′;
R^{a}′, R^{b}′, and R^{c}′ at each occurrence are independently halogen, —CN, nitro, N₃, —SO₂NH₂, or —Y—R^{Y};
Y is bond, O, NH, C(O), OC(O), C(O)NH, or S; and
R^{Y} is H, alkyl, alkenyl, alkynyl, alkoxy, cycloalkyl, aryl, heteroaryl, or heterocyclyl, wherein R^{Y} is optionally substituted;
provided that the compound is not N-(3-methyl-1-oxo-1-(thiazol-2-ylamino)butan-2-yl)thiophene-2-carboxamide.

2. The compound of claim 1, wherein the compound is compound of formula (I′-a), or a pharmaceutically acceptable salt thereof.

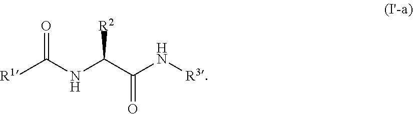

3. The compound of claim 1, wherein R¹′ is

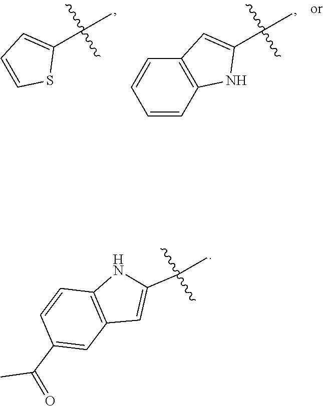 [SMILES: *c1cccs1 ; *c1cc2ccccc2[nH]1 ; *c1cc2cc(C(C)=O)ccc2[nH]1]

4. The compound of claim 1, wherein R²′ is C₂₋₄alkyl or benzyl.

5. The compound of claim 1, wherein R³′ is

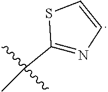 [SMILES: *c1nccs1]

6. The compound of claim 1, wherein the compound is selected from the group consisting of 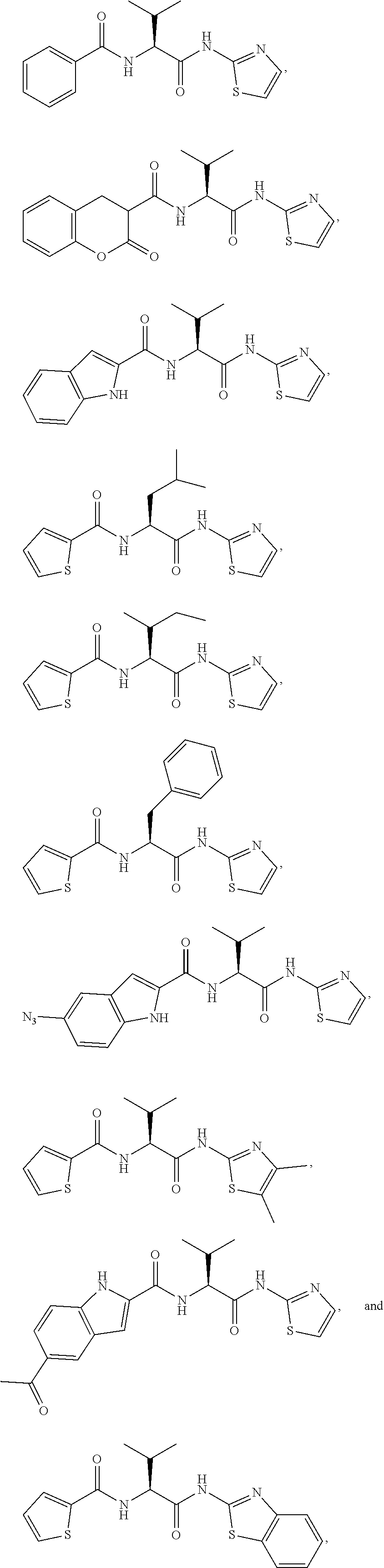
or a pharmaceutically acceptable salt thereof.

7. The compound of claim 1, wherein the compound is selected from the group consisting of 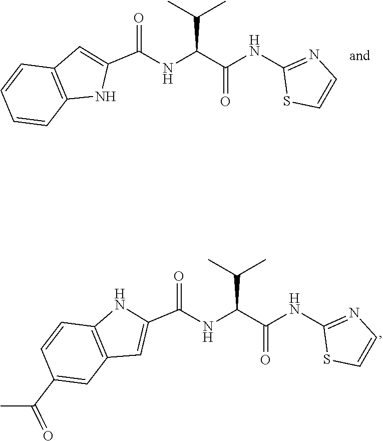 [SMILES: CC(C)[C@H](NC(=O)c1cc2ccccc2[nH]1)C(=O)Nc1nccs1 ; CC(=O)c1ccc2[nH]c(C(=O)N[C@H](C(=O)Nc3nccs3)C(C)C)cc2c1]
or a pharmaceutically acceptable salt thereof.

8. A compound, which is an enantiomerically enriched 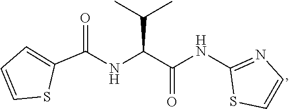 [SMILES: CC(C)[C@H](NC(=O)c1cccs1)C(=O)Nc1nccs1]
or a pharmaceutically acceptable salt thereof.

9. The compound of claim 8, wherein the compound has an enantiomeric excess (ee) value of at least 90%.

10. A pharmaceutical composition comprising a compound of claim 1, or a pharmaceutically acceptable salt thereof, and a pharmaceutically acceptable carrier.

11. A method for inhibiting a heat shock protein 70 (HSP70) comprising contacting the HSP70 with a pharmaceutically effective amount of a compound of formula (I), or a pharmaceutically acceptable salt thereof, 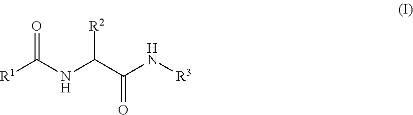
wherein
R¹ is an aryl or a heteroaryl, wherein R¹ is optionally substituted with one or more R^{a};
R² is C₁₋₆alkyl, aryl, heteroaryl, C₁₋₄alkylene-aryl, or C₁₋₄alkylene-heteroaryl, wherein R² is optionally substituted with one or more R^{b};
R³ is an aryl or a heteroaryl, wherein R³ is optionally substituted with one or more R^{c};
R^{a}, R^{b}, and R^{c} at each occurrence are independently halogen, —CN, nitro, N₃, —SO₂NH₂, or —X—R^{X},
X is bond, O, NH, C(O), OC(O), C(O)NH, or S; and
R^{X} is H, alkyl, alkenyl, alkynyl, alkoxy, cycloalkyl, aryl, heteroaryl, or heterocyclyl, wherein R^{X} is optionally substituted.

12. The method of claim 11, wherein the compound is compound of formula (I-a), or a pharmaceutically acceptable salt thereof,

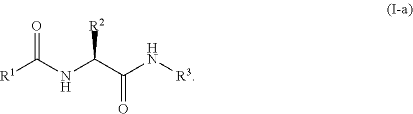

13. The method of claim 11, wherein R¹ is

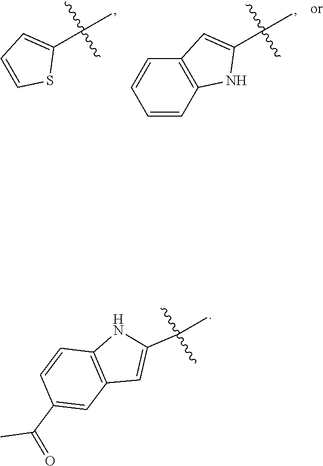 [SMILES: *c1cccs1 ; *c1cc2ccccc2[nH]1 ; *c1cc2cc(C(C)=O)ccc2[nH]1]

14. The method of claim 11, wherein R² is C₂₋₄alkyl or benzyl.

15. The method of claim 11, wherein R³ is

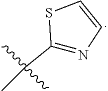 [SMILES: *c1nccs1]

16. The method of claim 11, wherein the compound is selected from the group consisting of 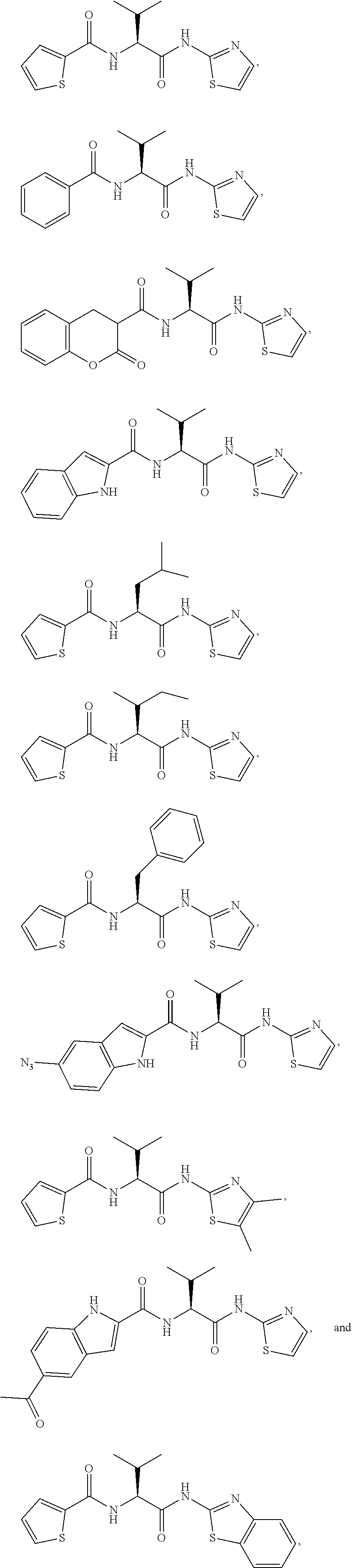
or a pharmaceutically acceptable salt thereof.

17. The method of claim 11, wherein the compound is selected from the group consisting of 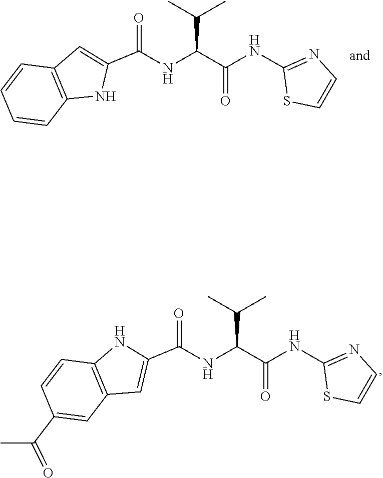 [SMILES: CC(C)[C@H](NC(=O)c1cc2ccccc2[nH]1)C(=O)Nc1nccs1 ; CC(=O)c1ccc2[nH]c(C(=O)N[C@H](C(=O)Nc3nccs3)C(C)C)cc2c1]
or a pharmaceutically acceptable salt thereof.

18. The method of claim 11, wherein the HSP70 comprises HSPA5.

19. The method of claim 11, further comprising contacting the HSP70 with an additional active agent.

20. The method of claim 19, wherein the additional active agent is CB5083.

21. A method for treating a disease characterized by overexpression of a heat shock protein 70 (HSP70) comprising administrating to a subject in need thereof a pharmaceutically effective amount of a compound of formula (I), or a pharmaceutically acceptable salt thereof, 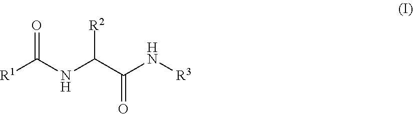
wherein
R¹ is an aryl or a heteroaryl, wherein R¹ is optionally substituted with one or more R^{a};
R² is C₁₀₋₆alkyl, aryl, heteroaryl, C₁₋₄alkylene-aryl, or C₁₋₄alkylene-heteroaryl, wherein R² is optionally substituted with one or more R^{b};
R³ is an aryl or a heteroaryl, wherein R³ is optionally substituted with one or more R^{c};
R^{a}, R^{b}, and R^{c} at each occurrence are independently halogen, —CN, nitro, N₃, —SO₂NH₂, or —X—R^{X},
X is bond, O, NH, C(O), OC(O), C(O)NH, or S; and
R^{X} is H, alkyl, alkenyl, alkynyl, alkoxy, cycloalkyl, aryl, heteroaryl, or heterocyclyl, wherein R^{X} is optionally substituted.

22. The method of claim 21, wherein the compound is compound of formula (I-a), or a pharmaceutically acceptable salt thereof,

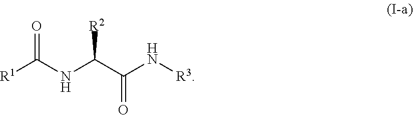

23. The method of claim 21, wherein R¹ is

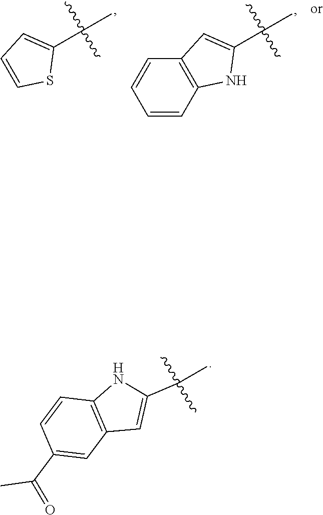 [SMILES: *c1cccs1 ; *c1cc2ccccc2[nH]1 ; *c1cc2cc(C(C)=O)ccc2[nH]1]

24. The method of claim 21, wherein R² is C₂₋₄alkyl or benzyl.

25. The method of claim 21, wherein R³ is

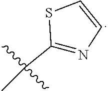 [SMILES: *c1nccs1]

26. The method of claim 21, wherein the compound is selected from the group consisting of 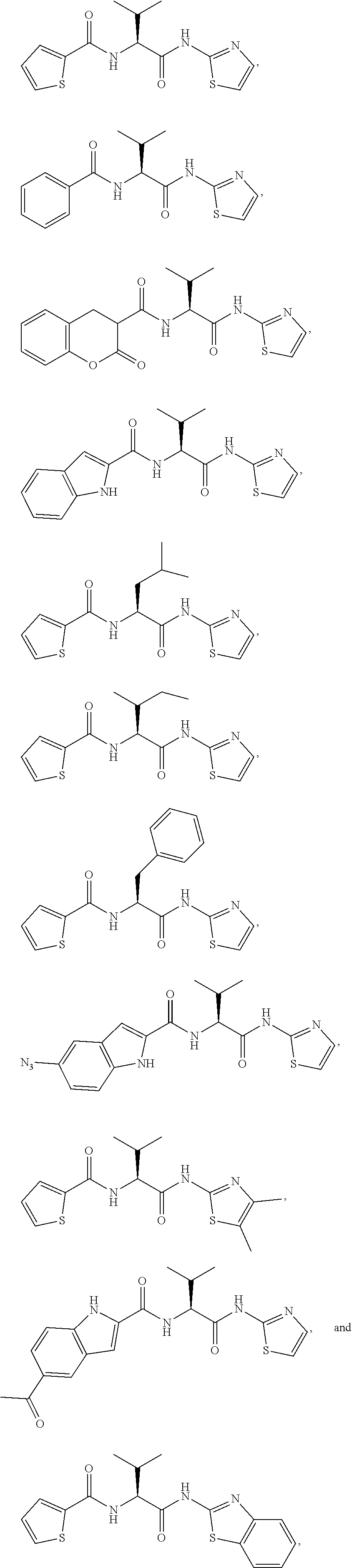
or a pharmaceutically acceptable salt thereof.

27. The method of claim 21, wherein the compound is selected from the group consisting of 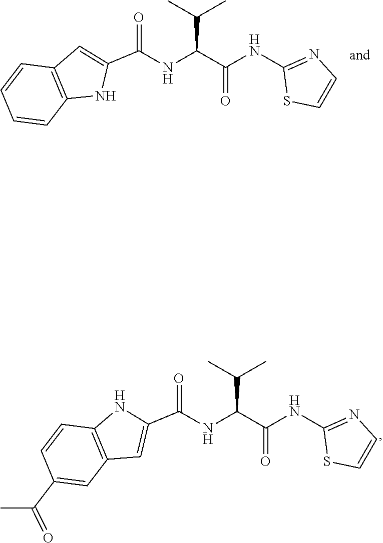 [SMILES: CC(C)[C@H](NC(=O)c1cc2ccccc2[nH]1)C(=O)Nc1nccs1 ; CC(=O)c1ccc2[nH]c(C(=O)N[C@H](C(=O)Nc3nccs3)C(C)C)cc2c1]
or a pharmaceutically acceptable salt thereof.

28. The method of claim 21, wherein the HSP70 comprises HSPA5.

29. The method of claim 21, wherein the disease is cancer.

30. The method of claim 29, wherein the cancer is bladder cancer, breast cancer, cervical cancer, colorectal cancer, esophageal cancer, glioblastoma, endometrial cancer, leukemia, liver cancer, lung cancer, mantle cell lymphoma, melanoma, multiple myeloma, oral cancer, ovarian cancer, prostate cancer, pancreatic cancer, renal cancer, stomach cancer, testicular cancer, thyroid cancer, or a combination thereof.

31. The method of claim 30, wherein the cancer is breast cancer, colorectal cancer, lung cancer, renal cancer, or a combination thereof

32. The method of claim 21, further comprising administrating to the subject an additional active agent.

33. The method of claim 32, wherein the additional active agent is CB5083.
